(19) United States
(12) Patent Application Publication
(10) Pub. No.: US 2008/0200458 A1
(43) Pub. Date: Aug. 21, 2008

(54) Methods and compositions for the treatment of body composition disorders

(76) Inventors: Joseph Barbosa, Lambertville, NJ (US); Simon D.P. Baugh, Ringoes, NJ (US); Qiang Han, Levittown, PA (US); Victoria K. Lombardo, Belle Mead, NJ (US); Huy Van Nguyen, Mays Landing, NJ (US); Praveen K. Pabba, Pennington, NJ (US); Kristen M. Terranova, Lawrenceville, NJ (US)

Correspondence Address: LEXICON PHARMACEUTICALS, INC., 8800 TECHNOLOGY FOREST PLACE, THE WOODLANDS, TX, 77381-1160, US

(21) Appl. No.: 12/015,728

(22) Filed: Jan. 17, 2008

Related U.S. Application Data
(60) Provisional application No. 60/880,990, filed on Jan. 18, 2007.

(51) Int. Cl. A61K 31/5377; C12N 9/99; A61K 31/47; A61K 31/519; A61K 31/437; A61K 31/502; A61P 3/00; A61K 31/4365; A61K 31/404; A61K 31/136; A61K 31/4164; A61K 31/343

(52) U.S. Cl. 514/234.5; 435/184; 514/310; 514/260.1; 514/303; 514/248; 514/265.1; 514/301; 514/415; 514/657; 514/313; 514/407; 514/469

(57) ABSTRACT

This invention relates to methods of treating, managing and preventing body composition disorders, and to compounds and pharmaceutical compositions useful in such methods.

This application claims priority to U.S. provisional application No. 60/880,990, filed Jan. 18, 2007, the entirety of which is incorporated herein by reference.

### 1. FIELD OF THE INVENTION

This invention relates to methods of treating obesity and other disorders, and to compounds and pharmaceutical compositions useful in such methods.

### 2. BACKGROUND

In mammals, de novo synthesis of fatty acids initially results in the formation of palmitic acid. This fatty acid, along with dietary linoleic acid and alpha-linolenic acid, is subsequently converted to a variety of monounsaturated fatty acids and polyunsaturated fatty acids (PUFAs). The formation of PUFAs is catalyzed by desaturase enzymes, including delta-9, delta-6 and delta-5 desaturases, which are named for the position of the double bond they help form. Nakamura, M. T. and Takayuki, Y. N., Annu. Rev. Nutr. 24:345-376 (2004).

Delta-5 desaturase (“Δ5-desaturase” or “Δ5 desaturase”) is encoded by the gene FADS1. The polynucleotide and amino acid sequences of human (GENBANK Accession Nos. NM_{—}013402 and NP_{—}037534) and murine (GENBANK Accession Nos. NM_{—}146094 and NP_{—}666206) FADS1 have been described, and its expression has been studied. See, e.g., Cho, H. P., et al., J. Biol. Chem. 274(52): 37335-37339 (1999)). And for over 20 years, researchers have looked for correlations between disease and the enzyme's expression and activity.

For example, it was reported in 1985 that both Δ6 and Δ5-desaturase activities were found to be higher in obese mice than in lean controls. Hughes, S. and York, D. A., Biochem. J. 225:307-313 (1985). But the bulk of published studies describe different results. See, e.g., Jump, D. B. and Clarke, S. D., Annu. Rev. Nutr. 19:63-90 (1999); Poisson, J.-P. G. and Cunnane, S. C., J. Nutr. Biochem. 2:60-70 (1991); Mimouni, V. and Poisson, J. P., Arch. Int. Physiol. Biochim. Biophys. 99:111-121 (1991); Igal, R. A., et al., Mol. Cell. Endocrinol. 77:217-227 (1991); El Boustani, S., et al., Metabolism 38:315-321 (1997); Holman, R. T., et al., Proc. Natl. Acad. Sci. USA 80:2375-2379 (1983). In one, researchers studying lean and obese Zucker rats found no difference in Δ6 activity and reduced Δ5-desaturase activity in the obese animals. Pan, D. A., et al., J. Nutr. 124:1555-1565 (1994).

The activity of Δ5-desaturase has also been studied in humans. Researchers studying the obese-prone Pima Indians reported “that both impaired insulin action and obesity are independently associated with reduced Δ5 desaturase activity,” and suggested that “[w]hile determining the mechanisms underlying this relationship is important for future investigations, strategies aimed at restoring ‘normal’ enzyme activities . . . may have therapeutic importance in the ‘syndromes of insulin resistance.’” Pan, D. A., et al., J. Clin. Invest. 96:2802-2808, 2802 (1995). “Put simply, the higher the Δ5 desaturase activity, the better the insulin action.” Pan, D. A., et al., J. Nutr. 124:1555-1565, 1561 (1994). Moreover, the “strong inverse correlation between the Δ5-desaturase activity and percentage of body fat” observed in the Pima Indian studies “is totally in keeping with the findings in the obesity-prone animal models.” Id. The finding is also consistent with results of other human studies. See, e.g., Decsi, T., et al., Lipids 35:1179-1184 (2000).

U.S. Pat. No. 6,759,208 (“the '208 patent”) describes a screening assay that reportedly can be used to identify “agents” that decrease or increase Δ5-desaturase activity. See, e.g., the '208 patent, col. 9, lines 39-42. The patent further suggests various diseases that might be treated using a compound that decreases or increases Δ5-desaturase activity. See, e.g., id., col. 11, lns. 41-61. But it does not teach whether the enzyme's activity should be increased or decreased in order to treat any of the diseases, nor does it identify any compounds that can be used to increase or decrease Δ5-desaturase activity.

Compounds that inhibit Δ5-desaturase activity have been reported. See, e.g., Obukowicz, M. G., et al., Biochem. Pharmacol. 55:1045-1058 (1998); Obukowicz, M. G., et al., JPET 287:157-166 (1998); Kawashima, H., et al., Biochim. Biophys. Acta. 1299:34-38 (1996); Kawashima, H., et al., Biosci. Biotech. Biochem. 60(10):1672-1676 (1996); Shimizu, S., et al., Lipids 26(7):512-516 (1991). One is sesamin, which has been studied in hypertensive rats. See, e.g., Matsumura, Y., et al., Biol. Pharm. Bull. 18(7):1016-1019 (1995); Matsumura, Y., et al., Biol. Pharm. Bull. 18(9):1283-1285 (1995); Matsumura, Y., et al., Biol. Pharm. Bull. 21(5):469-473 (1998). Sesamin and related compounds are reportedly useful for the treatment of infection (see, e.g., U.S. Pat. No. 5,762,935) and inflammation (see, e.g., U.S. Pat. Nos. 6,107,334; 6,172,106), while others have claimed their use in the commercial manufacture of PUFAs (see, e.g., U.S. Pat. Nos. 5,093,249; 5,336,496; 5,376,541; and 6,280,982). Other, non-sesamin based compounds have also been studied. See, e.g., Obukowicz, M. G., et al., Biochem. Pharmacol. 55:1045-1058 (1998); Obukowicz, M. G., et al., JPET 287:157-166 (1998).

### 3. SUMMARY OF THE INVENTION

This invention is directed, in part, to compounds of formula I:

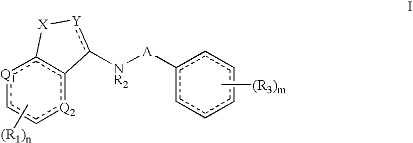

the substituents of which are defined herein, and pharmaceutically acceptable salt or solvate thereof.

Another embodiment encompasses compounds of formula II:

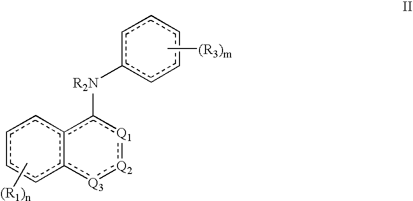

the substituents of which are defined herein, and pharmaceutically acceptable salt or solvate thereof.

Another embodiment encompasses compounds of formula III:

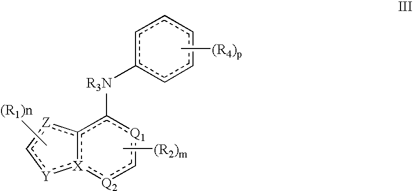

the substituents of which are defined herein, and pharmaceutically acceptable salt or solvate thereof.

Another embodiment encompasses pharmaceutical compositions comprising compounds of the invention (i.e., compounds disclosed herein).

Another embodiment encompasses a method of inhibiting Δ5-desaturase activity, which comprises contacting Δ5-desaturase with a compound of the invention.

Another embodiment encompasses methods of treating, preventing and managing body composition disorders (e.g., obesity, diabetes), which comprise administering to a patient in need of such treatment, prevention or management an effective amount of a compound of the invention.

### 4. BRIEF DESCRIPTION OF THE FIGURES

Certain aspects of this invention can be understood with reference to the following figures:

FIG. 1 shows the effect of a Δ5-desaturase inhibitor of the invention on the body weight of diet-induced obesity mice. The inhibitor was mixed with the mice's food.

### 5. DETAILED DESCRIPTION

This invention results, in part, from studies of FADS1 gene-disrupted mice. Those studies revealed that, contrary to suggestions in the literature, genetically engineered mice that do not express a functional product of the murine ortholog of the FADS1 gene exhibit decreased body fat and blood sugar as compared to their wild-type litter-mates.

In view of this discovery, compounds were developed that inhibit Δ5-desaturase. Particular compounds are believed to be useful for the treatment body composition disorders, such as obesity and diabetes.

### 5.1. Definitions

Unless otherwise indicated, the term “alkenyl” means a straight chain, branched and/or cyclic hydrocarbon having from 2 to 20 (e.g., 2 to 10 or 2 to 6) carbon atoms, and including at least one carbon-carbon double bond. Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

Unless otherwise indicated, the term “alkyl” means a straight chain, branched and/or cyclic (“cycloalkyl”) hydrocarbon having from 1 to 20 (e.g., 1 to 10 or 1 to 4) carbon atoms. Alkyl moieties having from 1 to 4 carbons are referred to as “lower alkyl.” Examples of alkyl groups include, but are not limited to, methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). The term “alkyl” includes saturated hydrocarbons as well as alkenyl and alkynyl moieties.

Unless otherwise indicated, the term “alkylaryl” or “alkyl-aryl” means an alkyl moiety bound to an aryl moiety.

Unless otherwise indicated, the term “alkylheteroaryl” or “alkyl-heteroaryl” means an alkyl moiety bound to a heteroaryl moiety.

Unless otherwise indicated, the term “alkylheterocycle” or “alkyl-heterocycle” means an alkyl moiety bound to a heterocycle moiety.

Unless otherwise indicated, the term “alkynyl” means a straight chain, branched or cyclic hydrocarbon having from 2 to 20 (e.g., 2 to 20 or 2 to 6) carbon atoms, and including at least one carbon-carbon triple bond. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl.

Unless otherwise indicated, the term “alkoxy” means an —O-alkyl group. Examples of alkoxy groups include, but are not limited to, —OCH₃, —OCH₂CH₃, —O(CH₂)₂CH₃, —O(CH₂)₃CH₃, —O(CH₂)₄—CH₃, and —O(CH₂)₅CH₃.

Unless otherwise indicated, the term “aryl” means an aromatic ring or an aromatic or partially aromatic ring system composed of carbon and hydrogen atoms. An aryl moiety may comprise multiple rings bound or fused together. Examples of aryl moieties include, but are not limited to, anthracenyl, azulenyl, biphenyl, fluorenyl, indan, indenyl, naphthyl, phenanthrenyl, phenyl, 1,2,3,4-tetrahydro-naphthalene, and tolyl.

Unless otherwise indicated, the term “arylalkyl” or “aryl-alkyl” means an aryl moiety bound to an alkyl moiety.

Unless otherwise indicated, the term “Δ5DIC₅₀” is the IC₅₀ of a compound determined using the in vitro liver microsomal assay described in the Examples, below.

Unless otherwise indicated, the terms “halogen” and “halo” encompass fluorine, chlorine, bromine, and iodine.

Unless otherwise indicated, the term “heteroalkyl” refers to an alkyl moiety (e.g., linear, branched or cyclic) in which at least one of its carbon atoms has been replaced with a heteroatom (e.g., N, O or S).

Unless otherwise indicated, the term “heteroaryl” means an aryl moiety wherein at least one of its carbon atoms has been replaced with a heteroatom (e.g., N, O or S). Examples include, but are not limited to, acridinyl, benzimidazolyl, benzofuranyl, benzoisothiazolyl, benzoisoxazolyl, benzoquinazolinyl, benzothiazolyl, benzoxazolyl, furyl, imidazolyl, indolyl, isothiazolyl, isoxazolyl, oxadiazolyl, oxazolyl, phthalazinyl, pyrazinyl, pyrazolyl, pyridazinyl, pyridyl, pyrimidinyl, pyrimidyl, pyrrolyl, quinazolinyl, quinolinyl, tetrazolyl, thiazolyl, and triazinyl.

Unless otherwise indicated, the term “heteroarylalkyl” or “heteroaryl-alkyl” means a heteroaryl moiety bound to an alkyl moeity.

Unless otherwise indicated, the term “heterocycle” refers to an aromatic, partially aromatic or non-aromatic monocyclic or polycyclic ring or ring system comprised of carbon, hydrogen and at least one heteroatom (e.g., N, O or S). A heterocycle may comprise multiple (i.e., two or more) rings fused or bound together. Heterocycles include heteroaryls. Examples include, but are not limited to, benzo[1,3]dioxolyl, 2,3-dihydro-benzo[1,4]dioxinyl, cinnolinyl, furanyl, hydantoinyl, morpholinyl, oxetanyl, oxiranyl, piperazinyl, piperidinyl, pyrrolidinonyl, pyrrolidinyl, tetrahydrofuranyl, tetrahydropyranyl, tetrahydropyridinyl, tetrahydropyrimidinyl, tetrahydrothiophenyl, tetrahydrothiopyranyl and valerolactamyl.

Unless otherwise indicated, the term “heterocyclealkyl” or “heterocycle-alkyl” refers to a heterocycle moiety bound to an alkyl moiety.

Unless otherwise indicated, the term “heterocycloalkyl” refers to a non-aromatic heterocycle.

Unless otherwise indicated, the term “heterocycloalkylalkyl” or “heterocycloalkyl-alkyl” refers to a heterocycloalkyl moiety bound to an alkyl moiety.

Unless otherwise indicated, the term “inhibits Δ5-desaturase in vivo” means the inhibition of Δ5-desaturase as determined using the in vivo assay described in the Examples, below.

Unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

Unless otherwise indicated, the term “MCHIC₅₀” is the IC₅₀ of a compound determined using the melanin concentrating hormone receptor assay described in the Examples, below.

Unless otherwise indicated, the term “pharmaceutically acceptable salts” refers to salts prepared from pharmaceutically acceptable non-toxic acids or bases including inorganic acids and bases and organic acids and bases. Suitable pharmaceutically acceptable base addition salts include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art. See, e.g., Remington's Pharmaceutical Sciences (18th ed., Mack Publishing, Easton Pa.: 1990) and Remington: The Science and Practice of Pharmacy (19th ed., Mack Publishing, Easton Pa.: 1995).

Unless otherwise indicated, a “potent Δ5-desaturase inhibitor” is a compound that has a Δ5DIC₅₀ of less than about 500 nM.

Unless otherwise indicated, the terms “prevent,” “preventing” and “prevention” contemplate an action that occurs before a patient begins to suffer from the specified disease or disorder, which inhibits or reduces the severity of the disease or disorder. In other words, the terms encompass prophylaxis.

Unless otherwise indicated, a “prophylactically effective amount” of a compound is an amount sufficient to prevent a disease or condition, or one or more symptoms associated with the disease or condition, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

Unless otherwise indicated, the term “stereoisomeric mixture” encompasses racemic mixtures as well as stereomerically enriched mixtures (e.g., R/S=30/70, 35/65, 40/60, 45/55, 55/45, 60/40, 65/35 and 70/30).

Unless otherwise indicated, the term “stereomerically pure” means a composition that comprises one stereoisomer of a compound and is substantially free of other stereoisomers of that compound. For example, a stereomerically pure composition of a compound having one stereocenter will be substantially free of the opposite stereoisomer of the compound. A stereomerically pure composition of a compound having two stereocenters will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound.

Unless otherwise indicated, the term “substituted,” when used to describe a chemical structure or moiety, refers to a derivative of that structure or moiety wherein one or more of its hydrogen atoms is substituted with an atom, chemical moiety or functional group such as, but not limited to, alcohol, aldehylde, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amidinyl (—C(NH)NH-alkyl or —C(NR)NH₂), amine (primary, secondary and tertiary such as alkylamino, arylamino, arylalkylamino), aroyl, aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carbonyl, carboxyl, carboxylic acid, carboxylic acid anhydride, carboxylic acid chloride, cyano, ester, epoxide, ether (e.g., methoxy, ethoxy), guanidino, halo, haloalkyl (e.g., —CCl₃, —CF₃, —C(CF₃)₃), heteroalkyl, hemiacetal, imine (primary and secondary), isocyanate, isothiocyanate, ketone, nitrile, nitro, oxygen (i.e., to provide an oxo group), phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) and urea (—NHCONH-alkyl-).

Unless otherwise indicated, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound means an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, the terms “treat,” “treating” and “treatment” contemplate an action that occurs while a patient is suffering from the specified disease or disorder, which reduces the severity of the disease or disorder, or retards or slows the progression of the disease or disorder.

Unless otherwise indicated, the term “include” has the same meaning as “include, but are not limited to,” and the term “includes” has the same meaning as “includes, but is not limited to.” Similarly, the term “such as” has the same meaning as the term “such as, but not limited to.”.

Unless otherwise indicated, one or more adjectives immediately preceding a series of nouns is to be construed as applying to each of the nouns. For example, the phrase “optionally substituted alky, aryl, or heteroaryl” has the same meaning as “optionally substituted alky, optionally substituted aryl, or optionally substituted heteroaryl.”

It should be noted that a chemical moiety that forms part of a larger compound may be described herein using a name commonly accorded it when it exists as a single molecule or a name commonly accorded its radical. For example, the terms “pyridine” and “pyridyl” are accorded the same meaning when used to describe a moiety attached to other chemical moieties. Thus, the two phrases “XOH, wherein X is pyridyl” and “XOH, wherein X is pyridine” are accorded the same meaning, and encompass the compounds pyridin-2-ol, pyridin-3-ol and pyridin-4-ol.

It should also be noted that if the stereochemistry of a structure or a portion of a structure is not indicated with, for example, bold or dashed lines, the structure or the portion of the structure is to be interpreted as encompassing all stereoisomers of it. Moreover, any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit.

### 5.2. Compounds

This invention encompasses compounds of formula I:

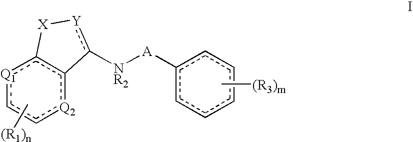

and pharmaceutically acceptable salts and solvates thereof, wherein: Q₁ is CR₁, CHR₁, N, or NR₁; Q₂ is CR₁, CHR₁, N, or NR₁; X is S, O, C(R₄R₅), or N(R₄); Y is C(R₄), C(R₄R₅), N, or N(R₄); A is a bond (i.e., NR₂ is directly attached to the optionally substituted phenyl moiety), CH₂, C(O), or SO₂; each R₁ is independently OR_{1A}, N(R_{1A})₂, NC(O)R_{1A}, hydrogen, cyano, nitro, halo, or optionally substituted alkyl, aryl, alkylaryl, arylalkyl, heterocycle, alkylheterocycle or heterocyclealkyl; each R_{1A} is independently hydrogen or optionally substituted alkyl, aryl, alkylaryl, arylalkyl, heterocycle, alkylheterocycle or heterocyclealkyl; R₂ is hydrogen or optionally substituted alkyl; each R₃ is independently OR_{3A}, N(R_{3A})₂, NC(O)R_{3A}, hydrogen, cyano, nitro, halo, or optionally substituted alkyl, aryl, alkylaryl, arylalkyl, heterocycle, alkylheterocycle or heterocyclealkyl; each R_{3A} is independently hydrogen or optionally substituted alkyl, aryl, alkylaryl, arylalkyl, heterocycle, alkylheterocycle or heterocyclealkyl; each R₄ is independently hydrogen or optionally substituted alkyl, aryl, alkylaryl, arylalkyl, heterocycle, alkylheterocycle or heterocyclealkyl; each R₅ is independently hydrogen or optionally substituted alkyl, aryl, alkylaryl, arylalkyl, heterocycle, alkylheterocycle or heterocyclealkyl; n is 1-4; and m is 1-5.

Another embodiment encompasses compounds of formula II:

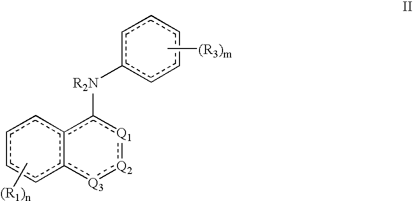

and pharmaceutically acceptable salts and solvates thereof, wherein: Q₁ is CR₄, CHR₄, N, or NR₄; Q₂ is CR₄, CHR₄, N, or NR₄; Q₃ is CR₄, CHR₄, N, or NR₄; each R₁ is independently OR_{1A}, N(R_{1A})₂, NC(O)R_{1A}, hydrogen, cyano, nitro, halo, or optionally substituted alkyl, aryl, alkylaryl, arylalkyl, heterocycle, alkylheterocycle or heterocyclealkyl; each R_{1A} is independently hydrogen or optionally substituted alkyl, aryl, alkylaryl, arylalkyl, heterocycle, alkylheterocycle or heterocyclealkyl; R₂ is hydrogen or optionally substituted alkyl; each R₃ is independently OR_{3A}, N(R_{3A})₂, NC(O)R_{3A}, hydrogen, cyano, nitro, halo, or optionally substituted alkyl, aryl, alkylaryl, arylalkyl, heterocycle, alkylheterocycle or heterocyclealkyl; each R_{3A} is independently hydrogen or optionally substituted alkyl, aryl, alkylaryl, arylalkyl, heterocycle, alkylheterocycle or heterocyclealkyl; each R₄ is independently hydrogen or optionally substituted alkyl, aryl, alkylaryl, arylalkyl, heterocycle, alkylheterocycle or heterocyclealkyl; n is 1-4; and m is 1-5; provided that at least one of Q₁, Q₂ and Q₃ is CHR₄ or CR₄.

Another embodiment encompasses compounds of formula III:

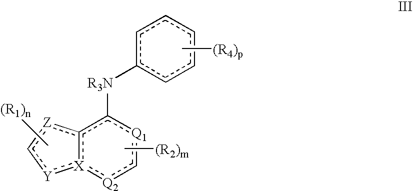

and pharmaceutically acceptable salts and solvates thereof, wherein: X is CH or N; Y is O, S, CR₁, CHR₁, N, or NR₁; Z is O, S, CR₁, CHR₁, N, or NR₁; Q₁ is CR₂, CHR₂, N, or NR₂; Q₂ is CR₂, CHR₂, N, or NR₂; each R₁ is independently OR_{1A}, N(R_{1A})₂, NC(O)R_{1A}, hydrogen, cyano, nitro, halo, or optionally substituted alkyl, aryl, alkylaryl, arylalkyl, heterocycle, alkylheterocycle or heterocyclealkyl; each R_{1A} is independently hydrogen or optionally substituted alkyl, aryl, alkylaryl, arylalkyl, heterocycle, alkylheterocycle or heterocyclealkyl; each R₂ is independently OR_{2A}, N(R_{2A})₂, NC(O)R_{2A}, hydrogen, cyano, nitro, halo, or optionally substituted alkyl, aryl, alkylaryl, arylalkyl, heterocycle, alkylheterocycle or heterocyclealkyl; each R_{2A} is independently hydrogen or optionally substituted alkyl, aryl, alkylaryl, arylalkyl, heterocycle, alkylheterocycle or heterocyclealkyl; R₃ is hydrogen or optionally substituted alkyl; each R₄ is independently OR_{4A}, N(R_{4A})₂, NC(O)R_{4A}, hydrogen, cyano, nitro, halo, or optionally substituted alkyl, aryl, alkylaryl, arylalkyl, heterocycle, alkylheterocycle or heterocyclealkyl; each R_{4A} is independently hydrogen or optionally substituted alkyl, aryl, alkylaryl, arylalkyl, heterocycle, alkylheterocycle or heterocyclealkyl; n is 1-3; m is 1-3; and p is 1-5.

Particular compounds are of formula I(a):

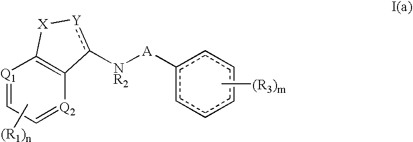

Others are of formula I(b):

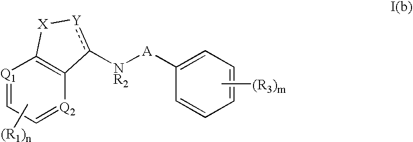

Others are of formula I(c):

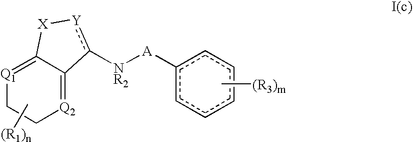

Others are of formula I(d):

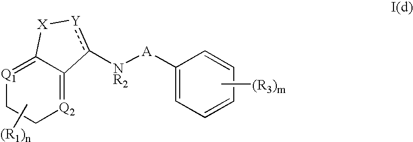

Others are of formula I(e):

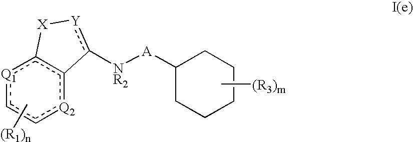

Others are of formula I(f):

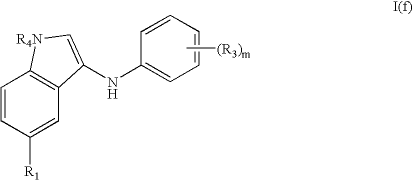

Others are of formula I(g):

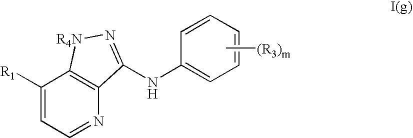

Others are of formula I(h):

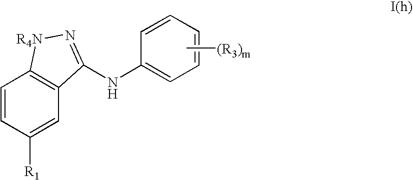

Particular compounds of formula I(h) are such that: 1) if R₁ is NH₂ or nitro, each R₃ is fluoro, and m is 1 or 2, then R₄ is not hydrogen; 2) if R₁ is methyl or chloro, R₃ is methyl or hydrogen, and m is 1, then R₄ is not hydrogen; 3) if R₁ is methyl, R₃ is para-chloro, and m is 1, then R₄ is not hydrogen; 4) if R₁ is halo, and each R₃ is hydrogen, then R₄ is not hydrogen; 5) if R₁ is NC(O)R_{1A}, then R₄ is not hydrogen; and 6) if R₁ is nitro, R₃ is cyano, and m is 1, then R₄ is not hydrogen.

Others are of formula I(i):

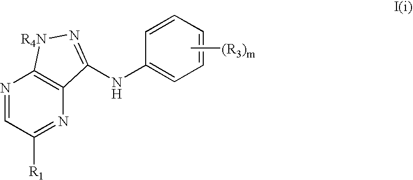

Others are of formula I(j):

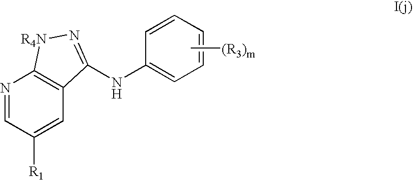

Others are of formula I(k):

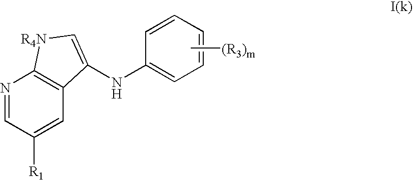

Others are of formula I(l):

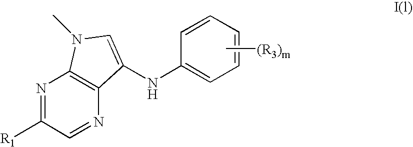

Others are of formula I(m):

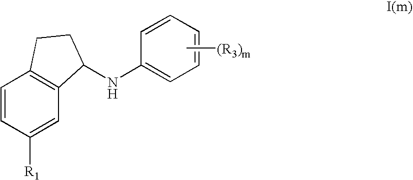

In particular compounds of formula I (e.g., I(a)-(m)) where applicable, Q₁ is CR₁. In others, Q₁ is N. In others, Q₂ is CR₁. In others, Q₂ is N. In others, both Q₁ and Q₂ are CR₁. In others, both Q₁ and Q₂ are N.

In others, X is C(R₄R₅)— In others, X is N(R₄)— In others, X is O. In others, X is S.

In others, Y is C(R₄) or C(R₄R₅). In others, Y is N or N(R₄). In others, X is N(R₄) or Y is C(R₄R₅) when both Q₁ and Q₂ are C.

In others, X is O and Y is N or NR₁.

In others, A is nothing. In others, A is CH₂. In others, A is C(O) or SO₂.

In others, R₁ is not hydrogen. In others, R₁ is halo or optionally substituted lower alkyl, aryl, or heteroaryl. In others, R₁ is optionally substituted lower alkyl. In others, R₁ is OR_{1A}. In others, R_{1A} is lower alkyl.

In others, n is 1 or 2. In others, n is 1.

In others, R₂ is hydrogen. In others, R₂ is lower alkyl.

In others, R₃ is halo or optionally substituted lower alkyl, aryl, or heteroaryl. In others, R₃ is optionally substituted lower alkyl. In others, R₃ is halo. In others, R₃ is OR_{3A}. In others, R_{3A} is lower alkyl.

In others, m is 1 or 2. In others, m is 1.

Certain compounds of the invention are of formula II(a):

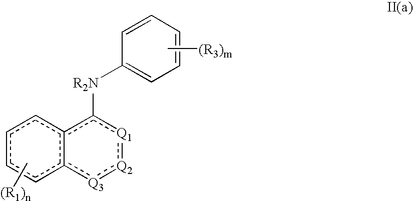

Others are of formula II(b):

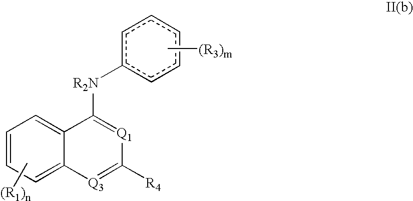

Others are of formula II(c):

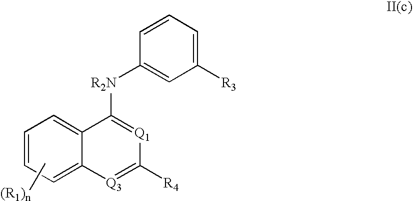

Others are of formula II(d):

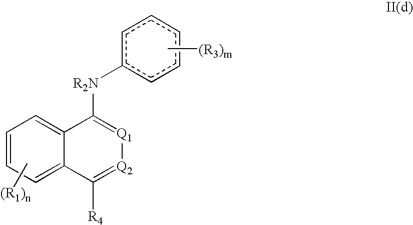

Others are of formula II(e):

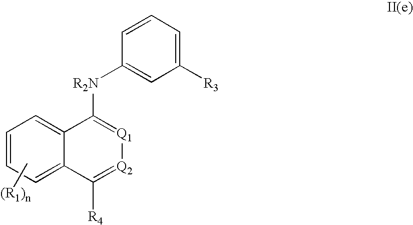

Others are of formula II(f):

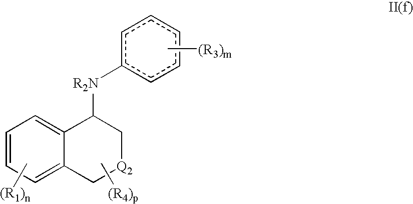

Others are of formula II(g):

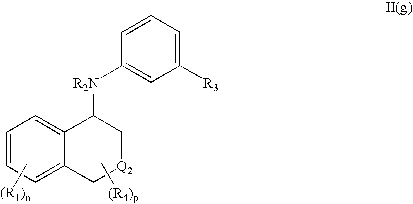

Others are of formula II(h):

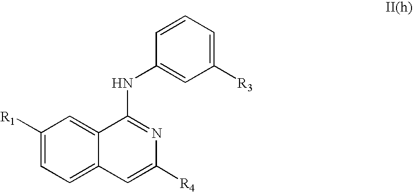

Certain compounds of formula II(h) are such that R₁ and R₄ are not both hydrogen.

Other compounds of the invention are of formula II(i):

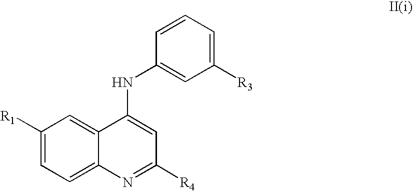

Certain compounds of formula II(i) are such that: 1) R₁ is not hydrogen; 2) R₁ is not methyl when R₄ is methyl; 3) R₁ is not halogen when R₃ is hydrogen or hydroxy; and/or 4) R₁ is not NH₂ if R₃ is hydroxy or NH₂.

Other compounds of the invention are of formula II(j):

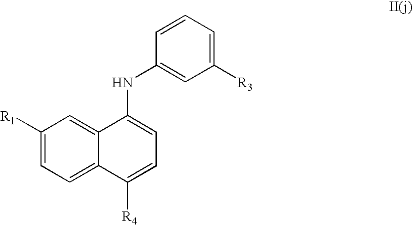

Certain compounds of formula II(j) are such that: 1) R₁ is not hydrogen; and/or 2) R₁ is not NH₂ when R₄ is hydrogen.

In particular compounds of formula II (e.g., II(a)-(j)), where applicable, Q₁ is NR₄. In others, Q₂ is NR₄. In others, Q₃ is NR₄. In others, R₄ is hydrogen. In others, Q₁ is NR₄ and Q₂ and Q₃ are both CR₄. In others, Q₁ and Q₃ are NR₄ and Q₂ is CR₄. In others, Q₁, Q₂ and Q₃ are all CHR₄ or CR₄.

In others, R₁ is hydrogen or halogen. In others, R₁ is OR_{1A}. In others, R_{1A} is hydrogen or optionally substituted lower alkyl.

In others, R₂ is hydrogen.

In others, R₃ is halogen or optionally substituted lower alkyl.

In others, n is 1.

In others, m is 1.

Certain compounds of the invention are of formula III(a):

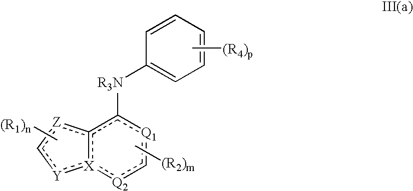

Others are of formula III(b):

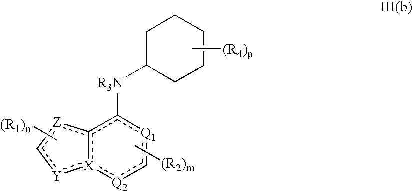

Others are of formula III(c):

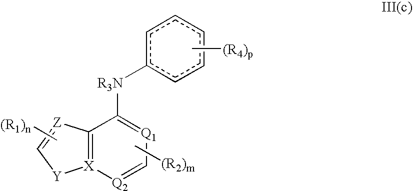

Others are of formula III(d):

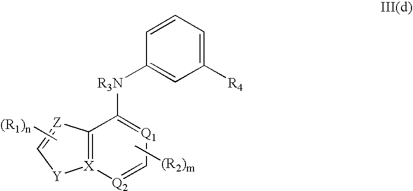

Others are of formula III(e):

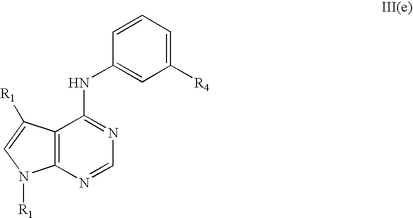

Certain compounds of formula III(e) are such that: 1) R₁ is not hydrogen; and/or 2) when R₁ is iodine, R₄ is not methyl.

Others are of formula III(f):

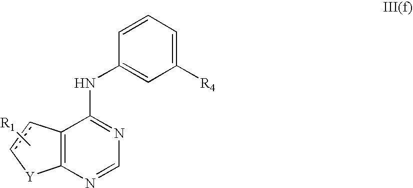

Certain compounds of formula III(f) are such that: 1) when Y is S, R₁ is not chloro; 2) when R₁ is hydrogen, R₄ is not hydrogen, halogen or methyl; and/or 3) when R₁ is methyl, R₄ is not hydrogen, chloro or methyl.

Others are of formula III(g):

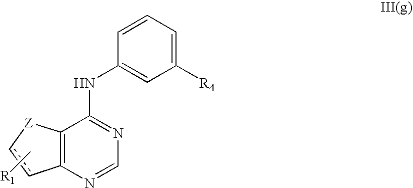

Certain compounds of formula III(g) are such that when G is S and R₁ is hydrogen, R₄ is not hydrogen or bromine.

Others are of formula III(h):

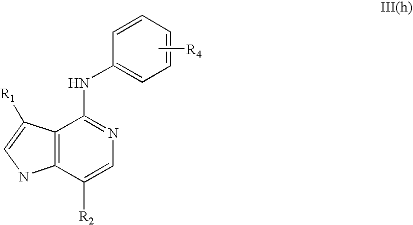

Others are of formula III(i):

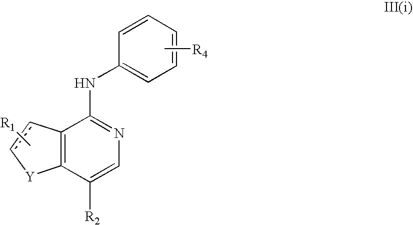

Certain compounds of formula III(i) are such that R₁ and R₂ are not both hydrogen when R₄ is not hydrogen.

Others are of formula III(j):

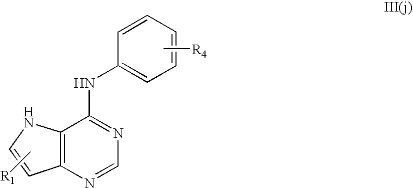

In particular compounds of formula III (e.g., III(a)-(j)), where applicable, X is CH. In others, Y is O. In others, Y is S. In others, Y is CHR₁. In others, Y is NR₁. In others, Z is CR₁. In others, Z is NR₁. In others, Z is N.

In others, Q₁ is CR₂. In others, Q₁ is N. In others, Q₂ is CR₂. In others, Q₂ is N.

In others, R₁ is hydrogen or optionally substituted lower alkyl. In others, R₁ is halogen.

In others, R₂ is hydrogen.

In others, R₃ is halogen or optionally substituted lower alkyl.

In others, n is 1.

In others, m is 1.

Preferred compounds are potent Δ5-desaturase inhibitors. For example, particular compounds have a Δ5DIC₅₀ of less than about 250, 150, 100, 50, 25 or 10 nM.

Certain compounds inhibit Δ5-desaturase in vivo by greater than about 75, 85 or 90 percent at about 60 mpk as determined using the in vivo assay described in the Examples, below.

Certain compounds of the invention do not significantly agonize the human melanin-concentrating hormone I (MCH₁) receptor. For example, certain compounds have a MCHIC₅₀ of greater than about 0.5, 1.0 or 2.0 μM.

### 5.3. Methods of Synthesis Compounds of the invention can be prepared by methods known in the art and by those described herein. For example, amide-linked compounds of formula I can be prepared by methods such as that shown below:

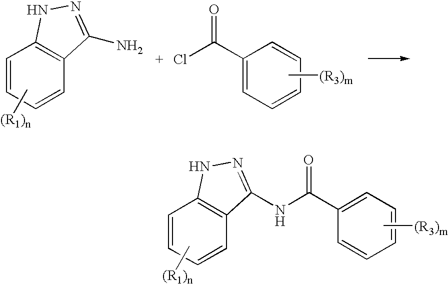

wherein, for example, the indazole amine is dissolved in a suitable solvent (e.g., dichloromethane), to which one equivalent base (e.g., triethylamine or pyridine) is added at room temperature. The acid chloride is then slowly added to the mixture, which is stirred for a time sufficient to provide the desired product.

To the extent it is not commercially available, the amine starting material (e.g., indazole amine) can be prepared by known methods:

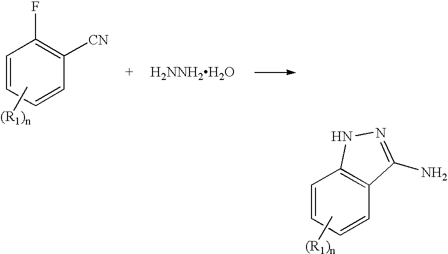

wherein the optionally substituted 2-fluoro benzonitrile and hydrazine monohydrate are combined in a suitable solvent (e.g., butanol) and heated (e.g., at 130° C.) in a sealed tube overnight. Substituted hydrazines can be used to obtain compounds such as N-methyl 1-methyl-1H-indazol-3-amine.

Amine-linked compounds of formula I can be prepared using approaches such as that shown below:

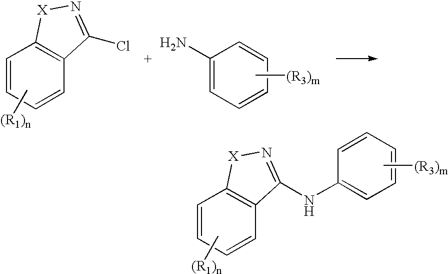

(e.g., wherein X is O, S or N) under conditions known in the art. For example, the 3-chloro indazole (X is NH) and the desired aniline salt are mixed in a sealed tube, and heated in an oil bath at a suitable temperature (e.g., 180° C.) overnight, or in a microwave (e.g., for 30 minutes), to afford the product after alkylation of the reaction mixture (e.g., with hot NaOH solution).

Other compounds of formula I can be prepared by methods such as that generally shown below:

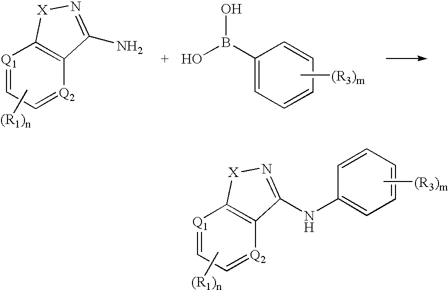

(e.g., wherein X is NR₄ or C(R₄R₅)) under conditions known in the art. For example, the amine is added to Cu(OAc)₂ in a suitable solvent (e.g., dichloromethane), followed by the boronic acid. The mixture is stirred for a suitable amount of time (e.g., five minutes), after which DIEA is added. The resulting mixture is stirred at room temperature for an amount of time sufficient (e.g., 14-18 hours) to provide the desired product. Base (e.g., 7N methanolic ammonia solution) is added, the mixture is stirred for an additional hour, and the product is isolated by conventional means.

Sulfonamide-linked compounds of formula I are readily prepared by methods such as that shown below:

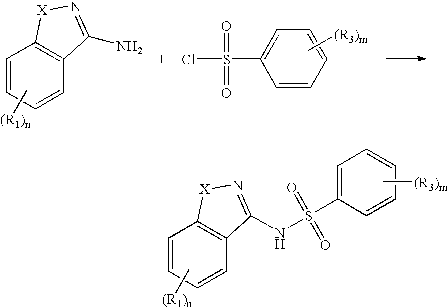

wherein the amine starting material and sulfonyl chloride are combined in a suitable solvent (e.g., pyridine) and stirred for a sufficient time (e.g., one hour). at a sufficient temperature (e.g., room temperature) to provide the desired product.

Compounds of formula II can also be prepared by methods known in the art. For example, quinaozline-based compounds can be obtained using methods such as that shown below:

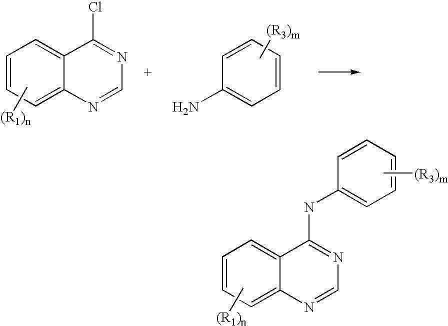

wherein the two reactants are combined in a suitable solvent (e.g., isopropanol), and the mixture is heated at a sufficient temperature (e.g., 80° C.) for a sufficient time (e.g., two hours) to provide the desired product. To the extent it is not commercially available, the quinazoline starting material can be prepared by methods such as that shown below:

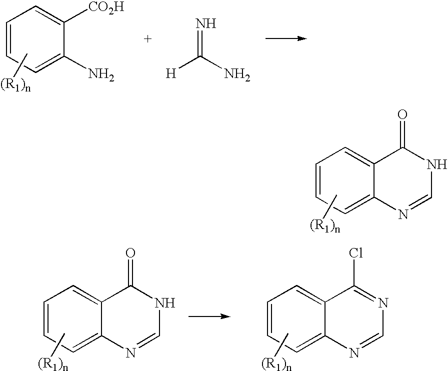

wherein the optionally substituted 2-amino benzoic acid is dissolved in a suitable solvent (e.g., ethanol), the formamidine acetate is added, and the resulting mixture is heated at reflux for an amount of time sufficient for the formation of the quinazolin-4(3H)-one (e.g., 16 hours). That intermediate is isolated, and then combined with POCl₃ to provide a mixture that is heated to reflux for an amount of time sufficient to form the 4-chloro-quinazoline product (e.g., six hours).

Additional compounds of formula II can be prepared by methods known in the art. For example, isoquinoline-based compounds can be obtained using methods such as that shown below:

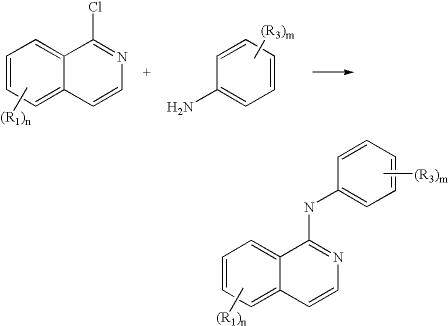

wherein the two reactants are combined in a suitable solvent (e.g., n-butanol), and the mixture is heated at a sufficient temperature (e.g., 80° C.) for a sufficient time (e.g., 6 hours) to provide the desired product. See, e.g., J. Med. Chem., 1999, 42, 3860-3873. To the extent it is not commercially available, the optionally substituted isoquinoline starting material can be prepared by methods such as that shown below:

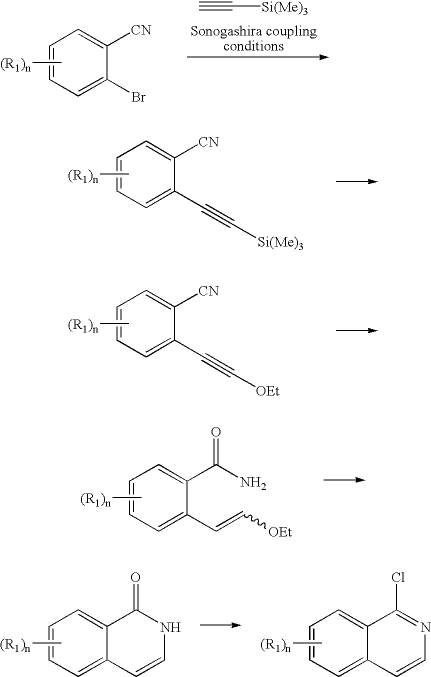

wherein the optionally substituted 2-bromobenzonitrile is combined with a trialkylsilylacetylene (e.g., TMS-acetylene), a palladium catalyst (e.g., PdCl₂(PPh₃)₂, Pd(PPh₃)₄), a copper catalyst (e.g., CuI), a base (e.g., Et₃N) (Sonogashira conditions outlined in J. Heterocyclic Chem., 1990, 1419-1424) in a suitable solvent (e.g., THF), under an inert atmosphere (e.g., N₂) and the resulting mixture is heated at an appropriate temperature (e.g., reflux) for an amount of time sufficient for the formation of the coupling product (e.g., 16 hours). That intermediate is isolated, and then combined with an alkoxide base (e.g., NaOEt) in a suitable solvent (e.g., EtOH) and the resulting mixture is heated at an appropriate temperature (e.g., reflux) for an amount of time sufficient for the formation of the enol ether (e.g., 16 hours). That intermediate is isolated, and combined with basic peroxide (e.g., Na₂CO₃ and H₂O₂) in a suitable solvent (e.g., acetone) and the resulting mixture is reacted at an appropriate temperature (e.g., room temperature to reflux) for an amount of time sufficient for the formation of the benzamide (e.g., 16-72 hours). That intermediate is isolated, and then combined with an acid catalyst (e.g., pTsOH) in a suitable solvent (e.g., benzene) and the resulting mixture is heated at an appropriate temperature (e.g., reflux) for an amount of time sufficient for the formation of the isoquinolin-1(2H)-one (e.g., 16 hours). That intermediate is isolated, and then combined with POCl₃ to provide a mixture that is heated to reflux for an amount of time sufficient to form the 1-chloroisoquinoline product (e.g., six hours). See, e.g., Bioorg. & Med. Chem. Lett., 2003, 11, 383-392.

Additional compounds of formula II can be prepared by methods known in the art. For example, quinoline-based compounds can be obtained using methods such as that shown below:

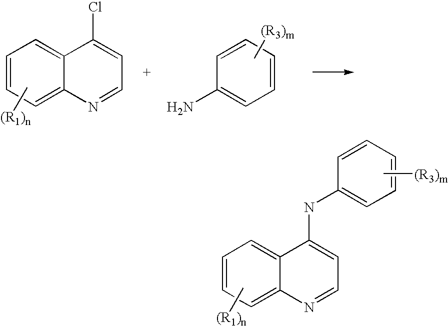

wherein the two reactants are combined in a suitable solvent (e.g., propanol), an acid catalyst (e.g., HCl) is added, and the mixture is heated at a sufficient temperature (e.g., 80° C.) for a sufficient time (e.g., 1-6 hours) to provide the desired product. See, e.g., J. Med. Chem., 2005, 48, 735-738. To the extent it is not commercially available, the optionally substituted 4-chloroquinoline starting material can be prepared by methods such as that shown below:

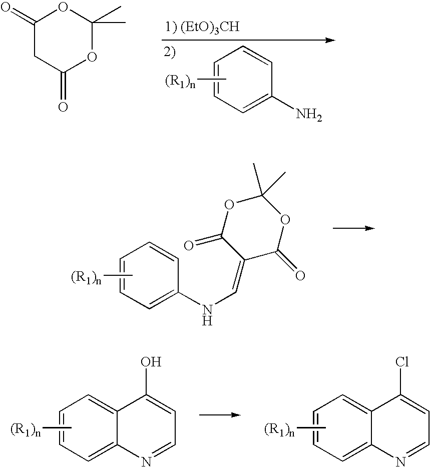

wherein Meldrum's Acid is combined with a trialkylorthoformate (e.g., triethyl orthoformate) and the resulting mixture is heated at an appropriate temperature (e.g., reflux) for an amount of time sufficient for the formation of the coupling product (e.g., 1-5 hours). The alkoxymethylene Meldrum's Acid that is formed in situ is combined with an optionally substituted aniline and an optional solvent (e.g., DMF), and the resulting mixture is heated at an appropriate temperature (e.g., reflux) for an amount of time sufficient for the formation of the coupling product (e.g., 2-4 hours). That intermediate is isolated, and then combined with a suitable solvent (e.g., diphenyl ether or Dowtherm) and the resulting mixture is heated at an appropriate temperature by microwave (e.g., 300° C.) or conventional (e.g., 250° C.) means for an amount of time sufficient for the formation of the quinolin-4-ol (e.g., 5-30 minutes). That intermediate is isolated, and then combined with POCl₃ to provide a mixture that is heated to reflux for an amount of time sufficient to form the 1-chloroquinoline product (e.g., 3-6 hours). See, e.g., Bioorg. & Med. Chem. Lett., 2004, 12, 731-3742.

Additional compounds of formula II can be prepared by methods known in the art. For example, napthyl-based compounds can be obtained using methods such as that shown below:

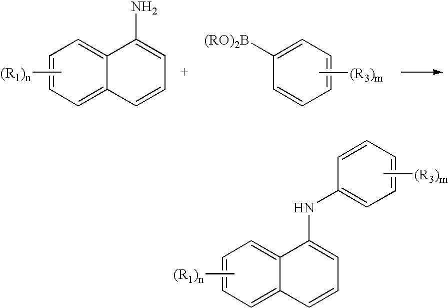

wherein the two reactants are combined in a suitable solvent (e.g., methylene chloride) with a copper catalyst (e.g., Cu(OAc)₂), a base is added (e.g., pyridine), and the mixture is stirred vigorously at an appropriate temperature (e.g., 25° C.) for a sufficient time (e.g., 16 hours) to provide the desired product. See, e.g., Org. Lett., 2001, 3, 2077-2079. To the extent it is not commercially available, the optionally substituted napthalen-1-amine starting material can be prepared by methods such as that shown below:

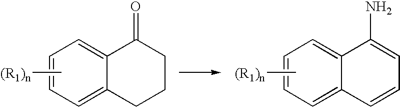

wherein a tetralone is combined with an optionally substituted benzylamine (e.g., benzylamine) and molecular sieves (e.g., 4 Å molecular sieves) in a suitable solvent (e.g., toluene), and the resulting mixture is heated at an appropriate temperature (e.g., reflux) for an amount of time sufficient for the formation of the coupling product (e.g., 1 hour). The resulting mixture is filtered, and the remaining molecular sieves are rinsed with an appropriate solvent (e.g., toluene). This solution is combined with a palladium catalyst (e.g., 10% palladium on carbon), placed in a sealed tube and heated to an appropriate temperature (e.g., 150° C.) for an amount of time sufficient for the formation of the napthalen-1-amine (e.g., 4 hours). See, e.g., Synthesis, 1993, 57-59.

Compounds of formula III can also be prepared by methods known in the art. For example, 7H-pyrrolo[2,3-d]pyrimidines-based compounds of formula III(e) can be obtained using methods such as that shown below:

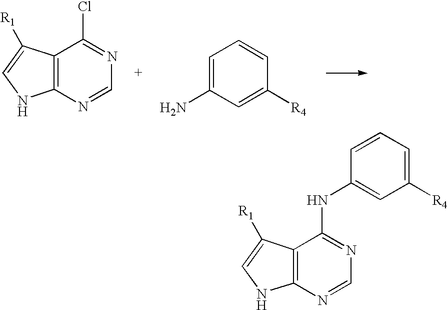

wherein the two reactants are combined in a suitable solvent (e.g., propanol), an acid catalyst (e.g., HCl) is added, and the mixture is heated at a sufficient temperature (e.g., 80° C.) for a sufficient time (e.g., 1-6 hours) to provide the desired product. To the extent it is not commercially available, the optionally substituted 4-chloro-7H-pyrrolo[2,3-d]pyrimidine starting material can be prepared by methods such as that shown below:

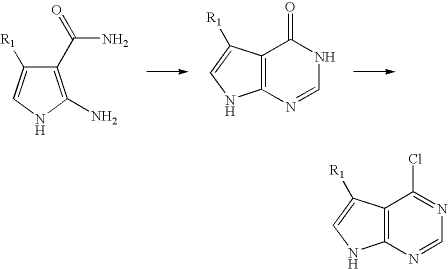

wherein the optionally substituted 2-amino-1H-pyrrole-3-carboxamide is combined with a trialkylorthoformate (e.g., triethyl orthoformate) and an acid catalyst (e.g., pTsOH) in a suitable solvent (e.g., DMF), and the mixture is stirred at a sufficient temperature (e.g., 25° C.) for a sufficient time (e.g., 16 hours) to provide the desired product. See, e.g., J. Med. Chem., 2005, 48, 7808-7820. That intermediate is isolated, and then combined with POCl₃ to provide a mixture that is heated to reflux for an amount of time sufficient to form the 4-chloro-7H-pyrrolo[2,3-d]pyrimidine product (e.g., 3-6 hours).

Additional compounds of formula III can be prepared by methods known in the art. For example, thieno[2,3-d]pyrimidine-4-amine-based compounds of formula III(f) can be obtained using methods such as that shown below:

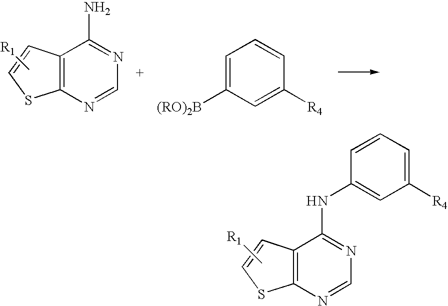

wherein the two reactants are combined in a suitable solvent (e.g., methylene chloride) with a copper catalyst (e.g., Cu(OAc)₂), a base is added (e.g., pyridine), and the mixture is stirred vigorously at an appropriate temperature (e.g., 25° C.) for a sufficient time (e.g., 16 hours) to provide the desired product. See, e.g., Tetrahedron. Lett., 1998, 39, 2933-2936. To the extent it is not commercially available, the optionally substituted thieno[2,3-d]pyrimidin-4-amine starting material can be prepared by methods such as that shown below:

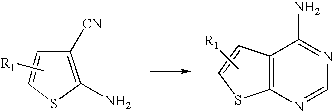

wherein the optionally substituted carbonyl-containing compound (e.g., ketone or aldehyde) is combined with malonyl nitrile, a sulfur source (e.g., S₈), a base (e.g., triethylamine), in a suitable solvent (e.g., DMF), and the mixture is stirred at an appropriate temperature (e.g., 25° C.) for a sufficient time (e.g., 16 hours) to provide the desired 2-aminothiophene-3-carbonitrile. See, e.g., J. Pharm. Sci., 2001, 90, 371-388. That intermediate is isolated, and then combined with a trialkylorthoformate (e.g., triethyl orthoformate) and an optional acid catalyst (e.g., AcOH), and the mixture is heated at a sufficient temperature (e.g., reflux) for a sufficient time (e.g., 1-2 hours) to provide the desired thieno[2,3-d]pyrimidin-4-amine. See, e.g., WO 2006/030031.

Compounds of formula III(g) can also be prepared by methods known in the art. For example, thieno[3,2-d]pyrimidin-4-amine-based compounds can be obtained using methods such as that shown below:

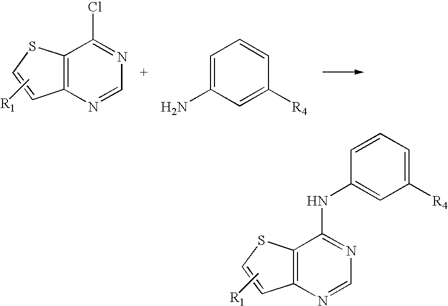

wherein the two reactants are combined in a suitable solvent (e.g., propanol), an acid catalyst (e.g., HCl) is added, and the mixture is heated at a sufficient temperature (e.g., 80° C.) for a sufficient time (e.g., 1-6 hours) to provide the desired product. To the extent it is not commercially available, the optionally substituted 4-chlorothieno[3,2-d]pyrimidine starting material can be prepared by methods such as that shown below:

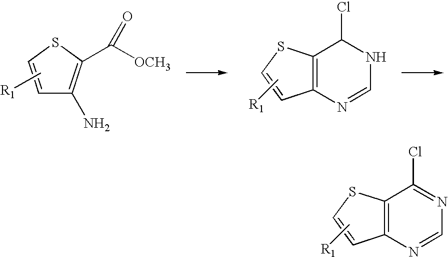

wherein the optionally substituted methyl 3-aminothiophene-2-carboxylate is combined with formic acid in an alkyl anhydride (e.g., acetic anhydride) and stirred at an appropriate temperature (e.g., 0-25° C.) for a sufficient time (e.g., 4 hours) to provide the desired product. See, e.g., US2006/0004002. That intermediate is isolated, and then combined with ammonium formate in formamide and the mixture is heated at a sufficient temperature (e.g., 160° C.) for a sufficient time (e.g., 1-6 hours) to provide the thieno[3,2-d]pyrimidin-4(3H)-one. That intermediate is isolated, and then combined with POCl₃ to provide a mixture that is heated to reflux for an amount of time sufficient to form the 4-chlorothieno[3,2-d]pyrimidine product (e.g., 3-6 hours).

Compounds of formula III(h) can also be prepared by methods known in the art. For example, 1H-pyrrolo[3,2-c]pyridin-4-amine-based compounds can be obtained using methods such as that shown below:

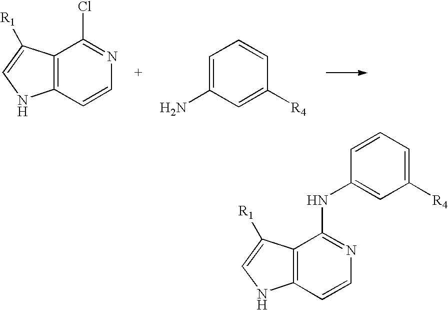

wherein the two reactants are combined in a suitable solvent (e.g., propanol), an acid catalyst (e.g., HCl) is added, and the mixture is heated at a sufficient temperature (e.g., 80° C.) for a sufficient time (e.g., 1-6 hours) to provide the desired product. To the extent it is not commercially available, the optionally substituted 4-chloro-1H-pyrrolo[3,2-c]pyridine starting material can be prepared by methods such as that shown below:

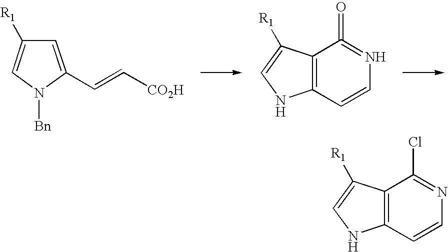

wherein the optionally substituted 3-(1-benzyl-1H-pyrrol-2-yl)acrylic acid is combined with a chloroformate (e.g., ethyl chloroformate), a suitable base (e.g., Et₃N), and sodium azide and stirred at an appropriate temperature (e.g., 0-25° C.) for a sufficient time (e.g., 4 hours) to provide the desired product. See, e.g., US2006/0004002.

Compounds of formula III(i) can also be prepared by methods known in the art. For example, furo[3,2-c]pyridin-4-amine-based compounds can be obtained using methods such as that shown below:

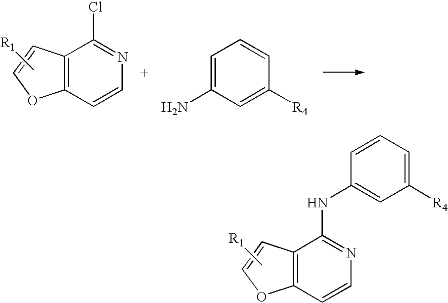

wherein the two reactants are combined in a suitable solvent (e.g., propanol), an acid catalyst (e.g., HCl) is added, and the mixture is heated at a sufficient temperature (e.g., 80° C.) for a sufficient time (e.g., 1-6 hours) to provide the desired product. To the extent it is not commercially available, the optionally substituted 4-chlorofuro[3,2-c]pyridine starting material can be prepared by methods such as that shown below:

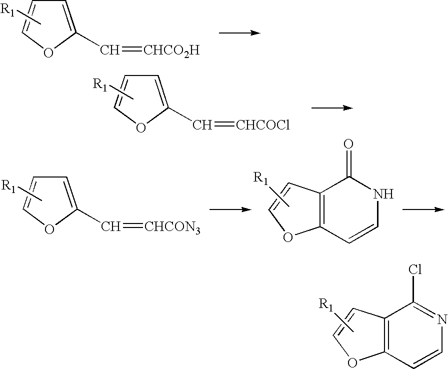

wherein an optionally substituted methyl 3-(furan-2-yl)acrylic acid is combined with a chlorinating agent (e.g., SOCl₂, DMF) in a suitable solvent (e.g., CHCl₃), and stirred and heated at a sufficient temperature (e.g., reflux) for a sufficient time (e.g., 1-6 hours) to provide the desired product. That intermediate is isolated, then combined with sodium azide in a suitable solvent (e.g., aqueous dioxane) and the resulting mixture is stirred at an appropriate temperature (e.g., 5° C.) for an amount of time sufficient for the formation of the 3-(furan-2-yl)acryloyl azide (e.g., 1 hour). That intermediate is isolated, combined with a suitable solvent (e.g., CH₂Cl₂) and added to a heated solution of a suitable solvent (e.g., (Ph)₂O) that is heated at a sufficient temperature (e.g., reflux) for a sufficient time (e.g., 1 hours) to provide the desired product. That intermediate is isolated, and then combined with POCl₃ to provide a mixture that is heated to reflux for an amount of time sufficient to form the 4-chlorofuro[3,2-c]pyridine product (e.g., 3-6 hours). See, e.g., J. Med. Chem., 1989, 32, 1147-1156.

Compounds of formula III(j) can also be prepared by methods known in the art. For example, 5H-pyrrolo[3,2-d]pyrimidin-4-amine-based compounds can be obtained using methods such as that shown below:

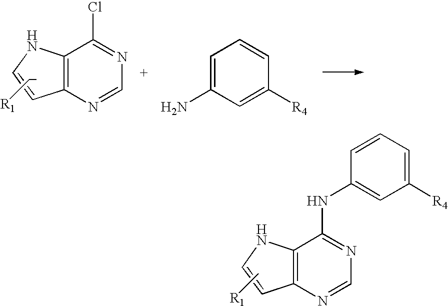

wherein the two reactants are combined in a suitable solvent (e.g., propanol), an acid catalyst (e.g., HCl) is added, and the mixture is heated at a sufficient temperature (e.g., 80° C.) for a sufficient time (e.g., 1-6 hours) to provide the desired product. To the extent it is not commercially available, the optionally substituted 4-chloro-5H-pyrrolo[3,2-d]pyrimidine starting material can be prepared by methods such as that shown below:

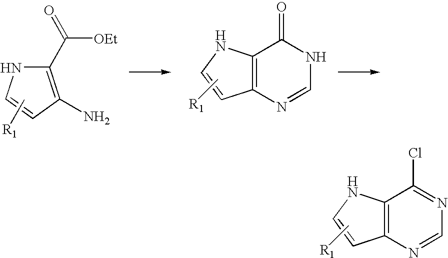

wherein the optionally substituted alkyl 3-amino-1H-pyrrole-2-carboxylate is combined with formamidine acetate in a suitable solvent (e.g., ethanol) and the mixture is heated at a sufficient temperature (e.g., reflux) for a sufficient time (e.g., 16 hours) to provide the desired product. See, e.g., J. Org. Chem., 1999, 64, 8411-8412. That intermediate is isolated, and then combined with POCl₃ to provide a mixture that is heated to reflux for an amount of time sufficient to form the 4-chloro-5H-pyrrolo[3,2-d]pyrimidine product (e.g., 3-6 hours).

### 5.4. Methods of Use

This invention encompasses a method of inhibiting Δ5-desaturase activity, which comprises contacting Δ5-desaturase with an effective amount of a compound of the invention (i.e., a compound disclosed herein). In one embodiment, the enzyme is in vivo. In another, it is ex vivo.

Another embodiment of the invention encompasses a method of treating, managing or preventing a metabolic or body composition disorder in a patient (e.g., a mammal, such as a human, dog or cat) in need of such treatment, management or prevention, which comprises inhibiting Δ5-desaturase in the patient to a degree sufficient to diminish, maintain or suppress the disorder or a symptom thereof. Examples of metabolic or body composition disorders include decreased glucose tolerance, diabetes (Type I or II), insulin resistance, non-insulin-dependent diabetes mellitus, obesity, and Syndrome X.

In one embodiment, the appetite of the patient is not significantly affected by the inhibition of Δ5-desaturase.

In one embodiment, the Δ5-desaturase is inhibited by administering to the patient a therapeutically or prophylactically effective amount of a Δ5-desaturase inhibitor (e.g., a potent Δ5-desaturase inhibitor). In another, the Δ5-desaturase is inhibited by administering to the patient an effective amount of a compound that disrupts or diminishes the expression of FADS1.

The amount, route of administration and dosing schedule of a compound may depend upon factors such as the specific indication to be treated, prevented or managed, and the age, gender and condition of the patient. The roles played by such factors are well known in the art, and may be accommodated by routine experimentation.

### 5.5. Pharmaceutical Formulations

This invention encompasses pharmaceutical compositions comprising one or more compounds of the invention. Certain pharmaceutical compositions are single unit dosage forms suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The formulation should suit the mode of administration. For example, oral administration requires enteric coatings to protect the compounds of this invention from degradation within the gastrointestinal tract. Similarly, a formulation may contain ingredients that facilitate delivery of the active ingredient(s) to the site of action. For example, compounds may be administered in liposomal formulations, in order to protect them from degradative enzymes, facilitate transport in circulatory system, and effect delivery across cell membranes to intracellular sites.

The composition, shape, and type of a dosage form will vary depending on its use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

5.5.1. Oral Dosage Forms

Pharmaceutical compositions of the invention suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See, e.g., Remington's Pharmaceutical Sciences, 18th ed., Mack Publishing, Easton Pa. (1990).

Typical oral dosage forms are prepared by combining the active ingredient(s) in an intimate admixture with at least one excipient according to conventional pharmaceutical compounding techniques. Excipients can take a wide variety of forms depending on the form of preparation desired for administration.

Because of their ease of administration, tablets and capsules represent the most advantageous oral dosage unit forms. If desired, tablets can be coated by standard aqueous or nonaqueous techniques. Such dosage forms can be prepared by conventional methods of pharmacy. In general, pharmaceutical compositions and dosage forms are prepared by uniformly and intimately admixing the active ingredients with liquid carriers, finely divided solid carriers, or both, and then shaping the product into the desired presentation if necessary. Disintegrants may be incorporated in solid dosage forms to facility rapid dissolution. Lubricants may also be incorporated to facilitate the manufacture of dosage forms (e.g., tablets).

5.5.2. Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are specifically sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

5.5.3. Transdermal, Topical and Mucosal Dosage Forms

Transdermal, topical, and mucosal dosage forms include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms are well known to those skilled in the pharmaceutical arts, and depend on the particular tissue to which a given pharmaceutical composition or dosage form will be applied.

Depending on the specific tissue to be treated, additional components may be used prior to, in conjunction with, or subsequent to treatment with active ingredients of the invention. For example, penetration enhancers may be used to assist in delivering active ingredients to the tissue.

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates may also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### 6. EXAMPLES

Aspects of this invention can be understood from the following examples, which do not limit its scope.

### 6.1. FADS1 Gene-Disrupted Mice

Mice homozygous (−/−) for the disruption of the FADS1 gene were generated by gene trapping, and were studied with mice heterozygous (+/−) for the disruption of the FADS1 gene and with wild-type (+/+) litter-mates. Disruption of the FADS1 gene was confirmed by RT-PCR (reverse transcription in conjunction with PCR).

The mice were subjected to a suite of medical diagnostic procedures. These studies revealed that the FADS1 knockout mice demonstrated an unexpected reduction in body weight with disproportional reduction in body fat, and displayed reduced glucose and insulin levels as compared to wild-type (+/+) controls. Subsequent tests on older animals on high (45%) fat diets revealed substantially improved glucose tolerance in FADS1 homozygous (−/−) knockout mice than in their wild-type litter-mates.

6.1.1. The Effect of FADS1 Disruption on Percent Body Fat

Mouse body-weight was determined to the nearest 0.1 gm using an Ohaus Scout scale. Body length was determined from nose to the base of tail. Body-weight and body length data were obtained for mice and female mice 16 weeks of age. The body-weight of eight homozygous (−/−) mice, four heterozygous (+/−) mice and four wild-type (+/+) mice was determined and analyzed. At this level and age, homozygous FADS1 (−/−) mice had a slightly reduced mean body-weight as compared to wild-type (+/+) control mice and heterozygous FADS1 (+/−) mice.

Body composition and percent body fat were measured by dual energy X-ray absorptiometry (DEXA) using a Piximus small animal densitometer (Lunar Corporation, Madison, Wis.). Individual mice were sedated with Avertin (1.25% solution, 2.5 mg/10 gm body weight delivered by intraperitoneal injection), immobilized on a positioning tray, and then placed on the Piximus imaging window. All scans were performed using the total body mode (0.18×0.18 mm), and the analyses was performed on the total body region of interest. The entire body, except the head, of each mouse was exposed for five minutes to a cone-shaped beam of both high and low energy X-rays. A high-resolution digital picture was taken of the image of the X-rays hitting a luminescent panel. Piximus software (version 1.45) calculated the ratio of attenuation of the high and low energies to separate bone from soft tissue compartments and, within the soft tissue compartment, to separate lean tissue mass from fat mass. Previous studies determined that this technique precisely measures fat and lean tissue mass, and that there is a close relationship between fat and lean tissue mass estimated by this technique with those measured using chemical carcass analysis. See Nagy, T. and Clair, A-L., Obesity Research 8:392-398 (2000).

Homozygous (−/−) FADS1 deficient mice displayed reduced body fat content as compared to their gender matched wild-type (+/+) litter-mates. An even greater reduction in percent body fat content was observed in female (−/−) mice as compared to gender match wild-type controls.

The trends towards reduced percent body fat in normal chow fed animals were further investigated by challenging additional FADS1 deficient animal cohorts with high fat diets (either 45 or 60 percent fat) beginning between 3-6 weeks of age. Comparisons of 36 homozygous FADS1 (−/−) deficient male animals with 36 wild-type litter-mates revealed a significant reduction in percent body fat in homozygous animals starting around 1-2 months after initiating the high fat diet. Similar results were observed when comparing 15 homozygous FADS1 (−/−) female animals to 17 female wild-type controls. After normalization for age, gender, and the type of high fat diet, the cumulative data revealed that homozygous FADS1 (−/−) male animals displayed a 40 percent reduction in body fat as compared to controls with only a nine percent reduction in lean body mass. A 27 percent reduction in percent body fat was observed in females, with a five percent reduction in lean body mass.

6.1.2. The Effect of FADS1 Disruption on Insulin Resistance

After an overnight fast, each mouse was bled. Blood was collected on an Accu-Check Advantage Test Strip, and the glucose concentration read using the Accu-Check Advantage Glucometer. Next, each mouse received a 20 percent glucose (20 gm/100 ml) solution to a final concentration of 2 mg glucose/gm body weight by oral gavage. Blood glucose was then measured on each mouse at 30, 60 and 90 minutes after the glucose administration by reopening the original wound.

The data was combined from four cohorts of mice that were on a 45 percent fat diet from 4-6 months. The ages of the mice at the time of glucose tolerance test (GTT) were between 22-29 weeks. Area under the GTT Curve: hom=20751+/−986 (n=49); WT−26592+/−1028 (43); p=9.2 E⁻⁵.

6.1.3. The Effect of FADS1 Disruption on Food Consumption

To determine if the observed alterations in body composition were the result of a loss of appetite or sickness, food consumption of male homozygous (−/−) FADS1 deficient mice and wild-type (+/+) litter-mates was accessed. This was done by housing groups of three mice of the same genotype in small rodent cages modified with metal adapter gates typically used in metabolic cages (Nalgene 650-0324). Stainless steel food baskets were used, which could hold up to 70 g of pelleted food and which contained a wire mesh with 1 cm² holes through which the mice could reach and gnaw the food pellets without being able to drag pieces for hoarding in the cage. The food baskets were weighed daily, and then topped off with food that was carefully arranged to allow for easy and continuous access. Spill from the food baskets was about 0.1 g per day, which represented an error of 2.5% of a mouse's total 24 hour food intake. The group housed mice were allowed to acclimatize to the cages to allow them to get used to eating off of the food hopper. Once the mice were acclimatized as observed by body weight stabilization, their daily food intake was measured for at least five days.

The food intake (expressed as grams of 45 percent fat diet consumed per mouse per day) of the male homozygous (−/−) FADS1 deficient mice (15 total—five cages of three mice each) was 3.4 g, compared to 3.1 g of food intake for the male wild-type (+/+) mice (9 total—three cages of three mice each). Therefore, the alterations in body composition observed in the FADS1 deficient mice were not the result of a loss of appetite or sickness.

6.1.4. The Effect of FADS1 Disruption on Glucose Tolerance

The effect of FADS1 disruption on glucose tolerance was determined by performing a glucose tolerance test (GTT) on 49 male homozygous (−/−) FADS1 deficient mice and 43 male wild-type (+/+) mice. The mice were 22-29 weeks of age, and had been on a 45 percent high fat diet for 4 to 6 months. After an overnight fast, the tail of each mouse was nicked once (at the lower end) with a surgical blade. Blood was collected on an Accu-Check Advantage Test Strip (Roche Diagnostics Corporation, Indianapolis, Ind.), and the glucose concentration read using an Accu-Check Advantage Glucometer (Roche Diagnostics Corporation). Each mouse then received a 20 percent glucose (20 g/100 ml) solution by oral gavage, for a final concentration of 2 mg glucose/gm body weight. Blood glucose was measured on each mouse 30, 60, and 90 minutes after the glucose administration by reopening the original wound by massaging the area, and removing the scab for subsequent bleedings.

The homozygous FADS1 deficient mice showed improved glucose tolerance at the 30, 60, and 90 minute time points. Before glucose administration, the homozygous male mice had an average blood glucose level of 90.3±23.5 mg/dL, compared to 96.5±28.1 mg/dL for the wild-type mice. At 30 minutes after the glucose administration, the homozygous male mice had an average blood glucose level of 318±98 mg/dL, compared to 393±107 mg/dL for the wild-type mice. At 60 minutes after the glucose administration, the homozygous male mice had an average blood glucose level of 240±102 mg/dL, compared to 333±98 mg/dL for the wild-type mice. At 90 minutes after the glucose administration, the homozygous male mice had an average blood glucose level of 180±87 mg/dL, compared to 222±75 mg/dL for the wild-type mice. The area under the GTT curve for the homozygous male mice was 20751±986, while the area under the GTT curve for the wild-type male mice was 26592±1028.

6.1.5. The Effect of FADS1 Disruption on Fasting Serum Lipids and Insulin

The effect of the FADS1 disruption on fasting serum lipid and insulin levels was determined in seven month old male mice that were weaned on a 45 percent high fat diet (15 homozygous FADS1 deficient and seven (insulin) or eight (serum lipid) wild-type mice). Blood was collected from overnight fasted mice in red-top tube with no additives. The blood was allowed to clot, spun down, and the serum was aliquoted in tubes for insulin and lipids analysis. Samples for insulin analysis were stored at −80° C., and then analyzed using a mouse insulin ELISA kit (Crystal Chem. Inc., Downers Grove, Ill.). Lipids were stored at 4-8° C., and then measured using a Cobas Integra 400 (Roche Diagnostics).

The homozygous FADS1 deficient mice showed a marked reduction in fasting serum cholesterol, triglyceride, and insulin levels compared to that of the wild-type mice. The serum cholesterol level of the homozygous FADS1 deficient mice was 153.5±40.1 mg/dl, compared to 248.4±74.1 for the wild-type mice (p=0.0006). The serum triglyceride level of the homozygous FADS1 deficient mice was 121.4±45.7 mg/dl, compared to 169.8±40.8 for the wild-type mice (p=0.0207). The serum insulin level of the homozygous FADS1 deficient mice was 0.58±0.40 ng/ml, compared to 1.80±1.48 for the wild-type mice (p=0.0067).

### 6.2. Synthesis of N-(3-chlorophenyl)isoquinolin-1-amine

To a stirred solution of 1-chloro-isoquinoline (0.050 g, 0.306 mmol) and 3-chloroaniline (0.078 g, 0.066 ml, 0.611 mmol) in isopropanol (2 ml) was added concentrated HCl (2 drops). The reaction was heated at 60° C. for 2 h, then cooled to room temperature and concentrated to dryness. The reaction was diluted with ethyl acetate (10 ml) and washed with saturated aqueous NaHCO₃ solution (3×5 ml). The organic extract was washed with brine (5 ml), dried (Na₂SO₄), and concentrated. The crude material was purified by column chromatography on silica gel (0-30% ethyl acetate/hexanes). The resulting material was purified by preparative TLC on silica (20% ethyl aceate/hexanes) to give the desired product (0.0297 g, 38%) as a beige solid.

¹H NMR (300 MHz, CD₃OD) δ ppm: 6.89 (d, J=6 Hz, 1H), 7.06 (d, J=6 Hz, 1H), 7.16 (t, J=8 Hz, 1H), 7.43 (d, J=7 Hz, 1H), 7.48 (d, J=8 Hz, 1H), 7.57 (t, J=8 Hz, 1H), 7.63-7.69 (m, 1H), 7.73 (t, J=2 Hz, 1H), 7.82 (d, J=6 Hz, 1H), 8.20 (d, J=8 Hz, 1H). HPLC: 100% at 1.225 minutes; Sunfire C18 4.6×50 mm; 10-90% methanol with 0.1% TFA: water; Gradient time=2 min; 3.5 ml/min; 254 nm. MS=255 M+H⁺.

### 6.3. Synthesis of N-(3-chlorophenyl)thieno[3,2-d]pyrimidin-4-amine

To a stirred solution of 4-chloro-4a,7a-dihydro-thieno[3,2-d]pyrimidine (0.050 g, 0.293 mmol) and 3-chloroaniline (0.041 g, 0.034 ml, 0.322 mmol) in isopropanol (0.748 ml) was added concentrated HCl (2 drops). The reaction was heated at 60° C. for 1 h, then cooled to room temperature and concentrated to dryness. The reaction was diluted with ethyl acetate (10 ml) and washed with saturated aqueous NaHCO₃ solution (3×5 ml). The organic extract was washed with brine (5 ml), dried (Na₂SO₄), and concentrated. The crude material was purified by column chromatography on silica gel (0-5% DCM/MeOH). The resulting material was recrystallized from ethyl acetate/hexanes to give the desired product (0.0295 g, 38%) as a white solid.

¹H NMR (300 MHz, CD₃OD) δ ppm: 7.16 (d, J=9 Hz, 1H), 7.36 (t, J=8 Hz, 2H), 7.43 (t, J=3 Hz, 1H), 7.68 (d, J=6 Hz, 1H), 7.98 (t, J=2 Hz, 1H), 8.12 (d, J=5 Hz, 1H), 8.61 (s, 1H). HPLC: 100% at 1.635 minutes; Sunfire C18 4.6×50 mm; 10-90% methanol with 0.1% TFA: water; Gradient time=2 min; 3.5 ml/min; 254 nm. MS=262 M+H⁺.

### 6.4. Synthesis of N-(3-chlorophenyl)imidazo[1,2-a]pyridin-8-amine

To a stirred solution of 8-bromo-imidazo[1,2-a]pyridine (0.064 g, 0.325 mmol) in toluene (0.650 ml) under N₂ was added 3-chloroaniline (0.050 g, 0.041 ml, 0.390 mmol), Pd₂(dba)₃ (0.001 g, 0.001 mmol), 2,2′-bis(diphenylphosphino)-1,1′-binaphthyl (0.002 g, 0.003 mmol), and sodium tert-butoxide (0.029 g, 0.455 mmol). The reaction was heated at 110° C. for 16 h and then cooled to room temperature. The mixture was diluted with ethyl acetate (1 ml) and H₂O (1 ml), and the phases were separated. The aqueous phase was extracted with ethyl acetate (3×1 ml). The combined organic extracts were washed with H₂O (1 ml), washed with brine (1 ml), dried (Na₂SO₄), and concentrated. The crude material was purified by column chromatography over silica gel (1-5% DCM/MeOH). The resulting material was recrystallized from ethyl acetate/hexanes to give the desired product (0.0086 g, 11%) as a gray gum.

¹H NMR (300 MHz, CD₃OD) δ ppm: 6.84 (ap t, J=7 Hz, 1H), 6.98-7.05 (m, 2H), 7.21-7.34 (m, 3H), 7.54 (bs, 1H), 7.84 (d, J=2 Hz, 1H), 8.01 (d, J=7 Hz, 1H). HPLC: 100% at 1.782 minutes; Sunfire C18 4.6×50 mm; 10-90% 10 mM ammonium acetate: acetonitrile; Gradient time=2 min; 3.5 ml/min; 254 nm. MS=244 M+H⁺.

### 6.5. Synthesis of N-(3-chlorophenyl)cinnolin-4-amine

To a stirred solution of 4-chloro-cinnoline (0.051 g, 0.310 mmol) and 3-chloroaniline (0.043 g, 0.036 ml, 0.341 mmol) in isopropanol (0.791 ml) was added concentrated HCl (2 drops). The reaction was heated at 60° C. for 1 h, then cooled to room temperature and concentrated to dryness. The reaction was diluted with ethyl acetate (10 ml) and washed with saturated aqueous NaHCO₃ solution (3×5 ml). The organic extract was washed with H₂O (5 ml), washed with brine (5 ml), dried (Na₂SO₄), and concentrated. The crude material was purified by column chromatography on silica gel (0-5% DCM/MeOH). The resulting material was recrystallized from ethyl acetate/hexanes to give the desired product (0.0137 g, 17%) as a yellow solid.

¹H NMR (300 MHz, DMSO) δ ppm: 7.23 (d, J=8 Hz, 1H), 7.35 (d, J=13 Hz, 2H), 7.46 (t, J=8.0 Hz, 1H), 7.69 (bs, 1H), 7.85 (t, J=8 Hz, 1H), 8.039 (bs, 1H), 8.34 (d, J=8 Hz, 1H), 8.64 (bs, 1H). HPLC: 96% at 1.470 minutes; Sunfire C18 4.6×50 mm; 10-90% methanol with 0.1% TFA: water; Gradient time=2 min; 3.5 ml/min; 254 nm. MS=256 M+H⁺.

### 6.6. Synthesis of N-(2,3-dihydro-1H-inden-4-yl)-7H-pyrrolo[2,3-d]pyrimidin-4-amine

To a stirred solution of 4-chloro-7H-pyrrolo[2,3-d]pyrimidine (0.075 g, 0.488 mmol) and 4-aminoindan (0.072 g, 0.537 mmol) in isopropanol (1 ml) was added concentrated HCl (2 drops). The reaction was heated at 60° C. for 5 h, then cooled to room temperature and concentrated to dryness. The reaction was diluted with ethyl acetate (5 ml) and washed with saturated aqueous NaHCO₃ solution (3×5 ml). The organic extract was washed with H₂O (5 ml), washed with brine (5 ml), dried (Na₂SO₄), and concentrated. The crude material was purified by column chromatography on silica gel (0-5% DCM/MeOH). The resulting material was recrystallized from ethyl acetate/hexanes to give the desired product (0.0060 g, 5%) as a white solid.

¹H NMR (300 MHz, CD₃OD) δ ppm: 2.06 (t, J=7 Hz, 2H), 2.82 (t, J=7 Hz, 2H), 2.99 (t, J=7 Hz, 2H), 6.29 (d, J=4 Hz, 1H), 7.09 (d, J=4 Hz, 1H), 7.16-7.23 (m, 3H), 8.10 (s, 1H). HPLC: 100% at 2.100 minutes; Sunfire C18 4.6×50 mm; 10-90% methanol: water with 0.1% TFA; Gradient time=2 min; 3.5 ml/min; 254 nm. MS=251 M+H⁺.

### 6.7. Synthesis of 4-(3-chlorophenylamino)thieno[3,2-c]pyridine-2-carbonitrile

A mixture of 2-bromo-thieno[3,2-c]pyridin-4-yl)-(3-chloro-phenyl)-amine (0.050 g, 0.147 mmol), Zn(CN)₂ (0.017 g, 0.147 mmol), and Pd(PPh₃)₄ (0.005 g, 0.004 mmol) in DMF (0.735 ml) was heated at 175° C. in the microwave for 2 minutes, cooled to room temperature, and heated at 175° C. in the microwave for an additional 20 minutes. The reaction was cooled to room temperature and diluted with ethyl acetate (5 ml). The organic phase was washed with H₂O (5 ml), dried (Na₂SO₄), and concentrated. The crude material was purified by column chromatography on silica (0-50% ethyl acetate/hexanes). The resulting material was purified by preparative-TLC (20% ethyl acetate:hexane). The resulting material was purified by preparative HPLC (10 mM aq ammonium acetate:acetonitrile) to give the desired product (0.0022 g, 5%) as a yellow solid.

¹H NMR (300 MHz, CD₃OD) δ ppm: 6.93 (d, J=6 Hz, 1H), 7.20 (t, J=8 Hz, 1H), 7.30 (d, J=3 Hz, 1H) 7.52 (d, J=9 Hz, 1H), 7.88 (t, J=2 Hz, 1H), 8.04 (d, J=6 Hz, 1H) 8.41 (s, 1H). HPLC: 100% at 2.177 minutes; Sunfire C18 4.6×50 mm; 10-90% 10 mM ammonium acetate: acetonitrile; Gradient time=2 min; 3.5 ml/min; 254 nm. MS=286 M+H⁺.

### 6.8. Synthesis of N¹-(3-chlorophenyl)-N⁶-(4-(dimethylamino)benzyl)-N⁶-methyl-2,3-dihydro-1H-indene-1,6-diamine

A mixture of (6-bromo-indan-1-yl)-(3-chloro-phenyl)-amine (50 mg, 1.55×10⁻⁴ mol), palladium acetate (1.7 mg, 7.73×10⁻⁶ mol), 2-(di-tert-butylphosphino)biphenyl (5 mg, 1.68×10⁻⁵ mol), and sodium tert-butoxide (16 mg, 1.62×10⁻⁴ mol) in toluene (0.4 ml) was treated with N,N-dimethyl-4-[(methylamino)methyl]aniline (26 mg, 1.55×10⁻⁴ mol). The reaction was heated at 80° C. for 10 h, and then cooled to 20° C. The reaction was diluted with ethyl acetate (15 ml) and water (10 ml). The aqueous layer was extracted with ethyl acetate (2×10 ml). The combined organic layers were washed with saturated aqueous NaCl (5 ml), dried (Na₂SO₄), and concentrated. The crude material was purified by preparative TLC on silica (10% ethyl acetate:hexane) to give the desired product (27 mg, 43%) as an orange gum.

¹H NMR (CDCl₃) δ ppm: 1.76-1.81 (m, 1H), 2.46-2.49 (m, 1H), 2.71-2.80 (m, 2H), 2.84 (s, 6H), 2.87 (s, 3H), 4.31 (s, 1H), 4.81 (t, J=6 Hz, 1H), 6.44-6.66 (m, 7H), 6.98-7.04 (m, 4H). HPLC: 96% at 2.743 minutes; Sunfire C18 4.6×50 mm; 10-90% acetonitrile: 10 mM ammonium acetate in water; Gradient time=2 min; 3.5 ml/min; 254 nm.

### 6.9. Synthesis of N-(3-chlorophenyl)-1-methyl-1H-indole-3-carboxamide

To a 0° C. suspension of N-methylindole-3-carboxylic acid (100 mg, 0.57 mmol) in CH₂Cl₂ (8 ml) was added N,N-dimethyl-4-aminopyridine (DMAP) (131 mg, 1.06 mmol), a solution of 3-chloroaniline (105 mg, 87 μl, 0.83 mmol) in CH₂Cl₂ (1.6 ml) and N-(3-dimethylaminopropyl)-N′-ethylcarbodiimide hydrochloride (EDCI.HCl) (123 mg, 0.65 mmol). The reaction was allowed to warm to 20° C., and was stirred at 20° C. for 16 h. The reaction was diluted with CH₂Cl₂ (15 ml), and was washed with dilute aqueous NaHCO₃ solution (2×15 ml). The organic layer was dried (Na₂SO₄), and concentrated. The crude mixture was purified by column on silica (10-50% ethyl acetate:hexane). The resulting material was purified by column on silica (5% methanol: CH₂Cl₂) to give the desired product (53 mg, 33%) as a white solid.

¹H NMR (CDCl₃) δ ppm 3.89 (s, 3H), 7.10-7.13 (m, 1H), 7.31-7.43 (m, 4H), 7.51-7.52 (m, 1H), 7.78 (bs, 1H), 7.80-7.82 (m, 2H), 8.03-8.06 (m, 1H). HPLC: 100% at 2.100 minutes; Sunfire C18 4.6×50 mm; 10-90% methanol with 0.1% TFA: water; Gradient time=2 min; 3.5 ml/min; 254 nm. MS=285 M+H⁺.

### 6.10. Synthesis of N-(3-chlorophenyl)-4-fluoronaphthalen-1-amine

To a mixture of 1-bromo-4-fluoronaphthalene (108 mg, 0.48 mmol) in toluene (1 ml) was added 3-chloroaniline (74 mg, 62 μl, 0.53 mmol), Pd₂(dibenzylideneacetone)₃ (1 mg, 0.001 mmol), BINAP (2 mg, 0.004 mmol), and sodium tert-butoxide (65 mg, 0.68 mmol). The reaction was heated at 110° C. for 16 h. The reaction was cooled to 20° C., diluted with ethyl acetate (15 ml), and this was washed with water (2×10 ml), and saturated aqueous NaCl (5 ml), dried (Na₂SO₄), and concentrated. The crude material was purified by preparative-TLC (10% ethyl acetate:hexane), followed by preparative-TLC (33% CH₂Cl₂: hexane) to give the desired product (41 mg, 31%) as an off-clear viscous liquid.

¹H NMR (CD₃OD) δ ppm: 6.71-6.83 (m, 3H), 7.07-7.18 (m, 2H), 7.21-7.35 (m, 1H), 7.54-7.63 (m, 2H), 8.05-8.12 (m, 2H). HPLC: 100% at 2.100 minutes; Sunfire C18 4.6×50 mm; 10-90% methanol: water with 0.1% TFA; Gradient time=2 min; 3.5 ml/min; 254 nm. MS=272 M+H⁺.

### 6.11. Synthesis of N-(3-chlorophenyl)-6-(trifluoromethyl)quinolin-4-amine

To a mixture of 4-chloro-6-trifluoromethylquinoline (72 mg, 0.31 mmol) and 3-chloroaniline (40 mg, 33, 0.31 mmol) in 2-propanol (0.75 ml) was added concentrated HCl (1 drop), and the reaction was heated at 70° C. for 1 h. The reaction was then cooled to 20° C., and concentrated to dryness. The residue was treated with water (15 ml) and saturated aqueous NaHCO₃ (2 ml). This mixture was extracted with ethyl acetate (2×15 ml). The combined organic layers were dried (Na₂SO₄), and concentrated. The crude material was purified by preparative-TLC (20% ethyl acetate:hexane). The resulting material was suspended in hexane (25 ml), heated, and filtered hot. This gave the desired product (7.2 mg, 7%) as a white solid.

¹H NMR (CDCl₃) δ ppm: 6.77 (bs, 1H), 7.09 (d, J=5 Hz, 1H), 7.26-7.19 (m, 2H), 7.37-7.33 (m, 2H), 7.87 (d, J=9 Hz, 1H), 8.16 (d, J=9 Hz, 1H), 8.25 (s, 1H), 8.69 (d, J=5 Hz, 1H). HPLC: 94% at 1.783 minutes; Sunfire C18 4.6×50 mm; 10-90% 10-90% methanol: water with 0.1% TFA; Gradient time=2 min; 3.5 ml/min; 254 nm. MS=323 M+H⁺.

### 6.12. Synthesis of N,1-diphenyl-1H-indazol-3-amine

To a round bottom flask, dry Cu(OAc)₂ powder (362 mg, 2.0 mmol, 1.0 equiv) and dichloromethane (3 ml) was added and stirred for 5 minutes. Then the indazole amine (266 mg, 2.0 mmol, 1.0 equiv) was added in one portion. To this reaction mixture phenyl boronic acid (244 mg, 2.0 mmol, 1.0 equiv) followed by Diisopropyl ethylamine (0.35 ml, 2.0 mmol, 1.0 equiv) were added one after the other. This reaction mixture was allowed to stir at room temperature for 24 hrs and then 2 ml of 6N NH₃ in methanol solution was added and stirred for additional 2 hrs. It is passed through a bed of silica gel and washed couple of times with dichloro methane solvent. The crude mixture was dried over MgSO₄ and concentrated. Preparative HPLC purification afforded 115 mgs (20% yield) of the bis substitution product and 168 mgs (40% yield) of the mono substitution product.

HPLC: Sunfire C18 4.6×50 mm×3.5 μM, Water: MeOH; 0.1% TFA, RT=2.60; LC-MS; M+H⁺=286.15.

### 6.13. Synthesis of 5-Methoxy-1H-indazol-3-ylamine

2-Fluoro-5-methoxy benzonitrile (70 mg, 0.46 mmol, 1.0 equiv.) and hydrazine monohydrate (0.3 ml, 6.0 mmol, 13.0 equiv.) were mixed in n-BuOH (2 ml) and heated the reaction mixture in a microwave oven at 180° C. for 45 mins. After it cooled to room temperature, the product indazole amine was precipitated (purified) by addition of n-hexane or n-heptane solvent. Further the precipitated solid was purified using MeOH:CH₂Cl₂ (1:1) solvent. The solid was washed with little water (to remove the excess hydrazine), and was dried for couple of hours to obtain nice crystalline white solid of indazole amine (46 mg) in 60% yield.

HPLC: Shim-pack VP ODS 4.6×50 m column, Water: MeOH; 0.1% TFA, RT=1.38; LC-MS; M+H⁺=164.2.

### 6.14. Synthesis of 5-Methoxy-1-(4-methoxy-benzyl)-1H-indazol-3-ylamine

In a round bottom flask, DMSO (50 ml) and ground KOH powder (1.365 g, 2.0 equiv.) were added and stirred for 5 minutes at room temperature. To this indazole amine (2.0 g, 1.0 equiv.) was added in one portion. After 5 minutes, 4-methoxy benzyl chloride (1.73 ml, 1.05 equiv.) was added using DMSO (25 ml) solvent over a period of 20-30 minutes. After stirring the reaction mixture for additional one hour, it was quenched with water and extracted the compound with dichloro methane (3×20 ml). The collected organic layer was washed with brine and passed through dry. Na₂SO₄. Evaporation of the solvent and silica gel column purification using 2-6% MeOH:CH₂Cl₂ solvent afforded the pure PMB protected product (2.4 g) in 70% yield.

HPLC: Shim-pack VP ODS 4.6×50 m column, Water: MeOH; 0.1% TFA, RT=2.80; LC-MS; M+H⁺=284.1.

### 6.15. Synthesis of (3-Chloro-phenyl)-5-methoxy-1-(4-methoxy-benzyl)-1H-indazol-3-ylamine

Cu(OAc)₂ powder (1.53 g, 8.45 mmol, 1.2 equiv) was added to a round bottom flask, to which dichloromethane (15 ml) and MeOH (1.0 ml) solvents were added. The mixture was stirred for 5 minutes, after which PMB protected indazole amine (2.0 g, 7.04 mmol, 1.0 equiv) was added in one portion. To this mixture, meta-chloro phenyl boronic acid (2.2 g, 14.1 mmol, 2.0 equiv) followed by di-isopropyl ethyl amine (1.5 ml, 8.45 mmol, 1.2 equiv) were added one after the other. This mixture was stirred at room temperature for 20 hrs, after which 6N NH₃ in methanol solution was added and stirred for additional 2 hrs. Then it was passed through a bed of silica gel and washed couple of times with dichloro methane solvent. The organic layer was washed with tartarate and brine solution. The crude mixture was dried over MgSO₄ and concentrated. ISCO purification using 2-5% MeOH:DCM solvent system afforded the pure product (2.1 g) in 75% yield.

HPLC: Sunfire C18 4.6×50 mm×3.5 uM, Water: MeOH; 0.1% TFA, RT=2.87; LC-MS; M+H⁺=394; (M+3)⁺=396.0.

### 6.16. Synthesis of N-(3-chlorophenyl)-5-methoxy-1H-indazol-3-amine

To the PMB protected indazole compound (70 mg, 0.17 mmol), trifluoro acetic acid (2.0 ml) was added in a round bottom flask and heated the reaction mixture at 65° C. for 90 minutes. After it cooled to room temperature, the TFA was evaporated under vacuum and diluted the reaction mixture with CH₂Cl₂ solvent. The organic layer was washed with NaHCO₃ solution followed by brine treatment produced the 94% pure (from HPLC) product. This mixture was purified again using silica gel chromatography using acetone and hexane solvents (20-40%) to afford 41 mg of the pure compound in 84% yield.

HPLC: YMC Pack ODS-A 3×50 mm, 7 um column, Water: MeOH; 0.1% TFA, RT=3.29; LC-MS; M+H⁺=274.1; (M+3)⁺=276.1.

### 6.17. Synthesis of 3-(3-chlorophenylamino)benzofuran-2-carboxamide

Cu(OAc)₂ powder (181 mg, 1.0 mmol, 1.0 equiv) and dichloromethane (3 ml) were added to a round bottomed flask and stirred for 5 minutes. Then, benzofuran carboxylic acid amide (176 mg, 1.0 mmol, 1.0 equiv) was added in one portion. To this mixture, m-chloro phenyl boronic acid (156 mg, 1.0 mmol, 1.0 equiv) followed by di-isopropyl ethylamine (0.17 ml, 1.0 mmol, 1.0 equiv) were added one after the other. This reaction mixture was allowed to stir at room temperature for 16 hrs and then 2 ml of 6N NH₃ in methanol solution was added and the mixture was stirred for an additional 8 hrs. It was passed through a bed of silica gel and washed couple of times with dichloro methane solvent. The crude mixture was dried over MgSO₄ and concentrated. Preparative HPLC purification afforded 120 mgs (40% yield) of the pure product.

HPLC: Sunfire C18 4.6×50 mm×3.5 uM, Water: MeOH; 0.1% TFA, RT=2.35; LC-MS; M+H⁺=287.05.

### 6.18. Synthesis of N,1-bis(4-methoxybenzyl)-5-nitro-1H-indazol-3-amine

To a solution of 5-nitro-1H-indazole-3-amine (1.78 g, 10 mmol, 1.0 equiv.) in DMSO (30 ml) was added finely ground KOH powder (0.561 g, 10 mmol, 1.0 equiv.). The mixture was stirred for 5 minutes at room temperature and PMB-Cl (1.50 ml, 1.05 equiv.) was added dropwise over a period of 30 minutes. After stirring for another 1 hr, the reaction mixture was quenched with water and extracted twice with dichloromethane. The combined organic extract was washed with brine, dried over Na₂SO₄ and concentrated in vacuo. Flash chromatography purification afforded mono-PMB protected compounds as well as the title product.

¹H NMR (CDCl₃) δ: 8.43 (1H, s), 8.05 (1H, d, J=9.3 Hz), 7.29 (2H, d, J=8.7 Hz), 7.08 (2H, d, J=8.7 Hz), 7.04 (1H, d, J=9.6 Hz), 6.80 (2H, d, J=9.6 Hz), 6.75 (2H, d, J=9.6 Hz), 5.25 (2H, s), 4.46 (2H, d, J=5.1 Hz), 4.37 (1H, br d, J=5.1 Hz), 3.72 (3H, s), 3.69 (3H, s); M+H⁺=419.

### 6.19. Synthesis of 2-fluoro-N-(5-methoxy-1H-indazol-3-yl)benzenesulfonamide

To a round bottom flask charged with the indazole amine (17 mg, 0.1 mmol, 1.0 equiv.) and 2-fluorobenzenesulfonyl chloride (20 mg, 0.1 mmol, 1.0 equiv.) was added 0.3 ml pyridine, and the resulting reddish brown solution was stirred at room temperature for 2 hrs. The reaction was concentrated in vacuo and preparative HPLC separation afforded the desired product as colorless crystals, 18 mg.

¹H NMR (acetone-d₆) δ: 9.38 (1H, brs), 7.78 (1H, t, J=5.7 Hz), 7.69-7.75 (1H, m), 7.40 (1H, s), 7.37 (1H, d, J=7.5 Hz), 7.30 (1H, t, J=7.8 Hz), 7.16 (1H, d, J=2.1 Hz), 7.02 (1H, dd, J=9.0, 2.4 Hz), 3.82 (3H, s); M+H⁺=322.

### 6.20. Synthesis of N-(5-methoxy-1H-indazol-3-yl)thiophene-2-sulfonamide

To a round bottom flask charged with the indazole amine (17 mg, 0.1 mmol, 1.0 equiv.) and 2-thiophenesulfonyl chloride (17 mg, 0.1 mmol, 1.0 equiv.) was added 0.3 ml pyridine, and the resulting reddish brown solution was stirred at room temperature for 1.5 hrs. The reaction was concentrated in vacuo and preparative HPLC separation afforded the desired product as colorless crystals, 25 mg.

¹H NMR (acetone-d₆) δ: 7.83 (1H, d, J=4.8 Hz), 7.54 (1H, d, J=3.6 Hz), 7.42 (1H, d, J=9.0 Hz), 7.12 (1H, s), 7.12 (1H, d, J=5.1 Hz), 7.04 (1H, dd, J=9.0, 2.4 Hz), 3.80 (3H, s); M+H⁺=310.

### 6.21. Synthesis of N-(3-chlorophenyl)-6-(4-(morpholinosulfonyl)-phenyl)quinazolin-4-amine

To a solution of 2-amino-5-bromo benzoic acid (2.16 g, 10 mmol, 1.0 equiv.) in 100 ml ethanol was added formamidine acetate (1.30 g, 12.5 mmol, 1.25 equiv.), and the reaction mixture was heated to reflux for 16 hrs. After the reaction was cooled to room temperature, the resulting white precipitate was collected via filtration and washed with water to afford 6-bromoquinazolin-4(3H)-one as a pale yellow prism, 1.78 g.

A suspension of 6-bromoquinazolin-4(3H)-one (1.45 g, 6.44 mmol, 1.0 equiv.) in 10 ml POCl₃ was heated to reflux for 6 hours. The resulting clear solution was then cooled to room temperature and concentrated in vacuo to afford 4-chloro-6-bromoquinazoline as an off-white crystal which was carried to the next step without further purification.

To the mixture of 4-chloro-6-bromoquinazoline (crude, 1.60 g) in isopropanol (20 ml) was added 3-chloroaniline (0.84 ml, 0.79 mmol, 1.2 equiv.). After heating to 80° C. for 2 hours, the reaction mixture was allowed to cool to room temperature and concentrated in vacuo. The resulting residue was diluted with 100 ml ethyl acetate, washed with sat. NaHCO₃ (aq.) and brine, dried over Na₂SO₄, and concentrated in vacuo. Flash chromatography purification afforded 6-bromoquinazoline as an off-white solid of 1 g.

To a 5 ml microwave vial was added 6-bromoquinazoline (34 mg, 0.1 mmol, 1.0 equiv.), N-Morpholinyl-4-boronbenzene sulfonylamide (27 mg, 0.1 mmol, 1.0 equiv.), Pd(PPh₃)₂Cl₂ (7 mg, 0.01 mmol, 0.1 equiv.) 2 ml acetonitrile and 0.3 ml aq. NaHCO₃ (1 M) were added and the reaction mixture was kept under microwave heating at 160° C. for 800 seconds. After cooling to the room temperature, the crude mixture was diluted with water and extracted twice with EtOAc. Preparative TLC purification afforded the desired product as a white solid of 9 mg.

¹H NMR (CDCl₃) δ: 8.84 (1H, brs), 8.36 (1H, br), 7.98-8.08 (4H, m), 7.75-7.90 (4H, m), 7.39 (1H, t, J=8.1 Hz), 7.21 (1H, d, J=8.1 Hz), 3.74 (4H, s), 3.04 (4H, s); M+H⁺=481.

### 6.22. Additional Compounds

Using synthetic methods known in the art and/or described herein, the following additional compounds were prepared:
6-(furan-3-yl)-N-phenylquinazolin-4-amine;
7-chloro-N-(3-chlorophenyl)quinazolin-4-amine;
1-(3-chlorophenyl)-3-(3,5-dimethylphenyl)urea;
N-(3-chlorophenyl)-6-methoxyquinazolin-4-amine;
4-(3-chlorophenylamino)quinazolin-6-ol;
tert-butyl 5-(3-chlorophenylamino)-3,4-dihydroisoquinoline-2(1H)-carboxylate;
4-(3-chlorophenylamino)quinazolin-6-yl acetate;
5-bromo-N-(3-chlorophenyl)-1-methyl-1H-indazol-3-amine;
N³-(3-chlorophenyl)-1H-indazole-3,5-diamine;
methyl 2-(3-(3-chlorophenylamino)-1H-indazol-5-ylamino)acetate;
N-(3-(3-chlorophenylamino)-1H-indazol-7-yl)acetamide;
N-(3-chlorophenyl)-7-(trifluoromethyl)-1H-indazol-3-amine;
N-(3-chlorophenyl)-6-fluoroquinazolin-4-amine;
(3-(3-chlorophenylamino)benzo[b]thiophen-2-yl)methanol;
N-(3-chlorophenyl)-7-ethyl-1H-indazol-3-amine;
5-bromo-N,1-bis(3-chlorophenyl)-1H-indazol-3-amine;
5-bromo-N-(3-chlorophenyl)-1H-indazol-3-amine;
N-(3-chlorophenyl)-7-nitro-1H-indazol-3-amine;
7-bromo-N-(3-chlorophenyl)-1H-indazol-3-amine;
3-(3-chlorophenylamino)-1H-indazol-5-ol;
(Z)-7-(but-2-en-2-yl)-N-(3-chlorophenyl)-1H-indazol-3-amine;
N-(3-chlorophenyl)benzo[d]isothiazol-3-amine;
N-(3-chlorophenyl)isoquinolin-1-amine;
N-(3-chlorophenyl)quinazolin-4-amine;
N-(3-chlorophenyl)-5-methyl-7H-pyrrolo[2,3-d]pyrimidin-4-amine;
N-(3-chlorophenyl)-7H-pyrrolo[2,3-d]pyrimidin-4-amine;
4-chloro-N-(3-chlorophenyl)phthalazin-1-amine;
N³-(3-chlorophenyl)-1H-indazole-3,7-diamine;
N-(3-chlorophenyl)-5-methyl-1H-indazol-3-amine;
N²-(3-chlorophenyl)-N⁶-methyl-N⁶-((R)-1-phenylethyl)-2,3-dihydro-1H-indene-1,6-diamine;
N-(3-chlorophenyl)-5-methoxy-1H-indazol-3-amine;
2-amino-N-m-tolylbenzamide;
5-amino-N-(3-chlorophenyl)-2-hydroxybenzamide;
N-(3-chlorophenyl)naphthalen-1-amine;
N-(3-chlorophenyl)isoquinolin-4-amine;
N-(3-chlorophenyl)-8-fluoroquinazolin-4-amine;
N³,N^{3′}-bis(3-chlorophenyl)-2,2′,3,3′-tetrahydro-1H,1′H-5,5′-biindene-3,3′-diamine;
N¹-(3-chlorophenyl)-N⁶-(2-methoxyethyl)-N⁶-methyl-2,3-dihydro-1H-indene-1,6-diamine;
N-(3-chlorophenyl)-5,7-difluoroquinazolin-4-amine;
N-(3-chlorophenyl)-6,7-difluoroquinazolin-4-amine;
N-(3-chlorophenyl)thieno[2,3-d]pyrimidin-4-amine;
N¹-(3-chlorophenyl)isoquinoline-1,3-diamine;
N-(3-chlorophenyl)thieno[3,2-d]pyrimidin-4-amine;
N¹-(3-chlorophenyl)-N⁶-(4-fluorobenzyl)-N⁶-methyl-2,3-dihydro-1H-indene-1,6-diamine;
N¹-(3-chlorophenyl)-N⁶-(3-methoxybenzyl)-N⁶-methyl-2,3-dihydro-1H-indene-1,6-diamine;
N-(3-chlorophenyl)-2-methoxy-7H-pyrrolo[2,3-d]pyrimidin-4-amine;
N-(3-chlorophenyl)furo[3,2-c]pyridin-4-amine;
N-(3-chlorophenyl)furo[3,2-c]pyridin-4-amine;
N¹-(3-chlorophenyl)-N⁶-(4-methoxyphenyl)-N⁶-methyl-2,3-dihydro-1H-indene-1,6-diamine;
N⁶-(4-methoxyphenyl)-N¹-(3-((4-methoxyphenyl)(methyl)amino)phenyl)-N⁶-methyl-2,3-dihydro-1H-indene-1,6-diamine;
(R)-6-chloro-N-(3-chlorophenyl)-2,3-dihydro-1H-inden-1-amine;
N¹-(3-chlorophenyl)-N⁶-methyl-N⁶-(4-morpholinobenzyl)-2,3-dihydro-1H-indene-1,6-diamine;
N¹-(3-chlorophenyl)-N⁶-(2,4-dimethoxybenzyl)-N⁶-methyl-2,3-dihydro-1H-indene-1,6-diamine;
N¹-(3-chlorophenyl)-N⁶-(2-(dimethylamino)ethyl)-N⁶-methyl-2,3-dihydro-1H-indene-1,6-diamine;
N-(3-chlorophenyl)-7-methyl-7H-pyrrolo[2,3-d]pyrimidin-4-amine;
N-(3-chlorophenyl)imidazo[1,2-a]pyridin-8-amine;
N-(3-chlorophenyl)cinnolin-4-amine;
4-(3-chlorophenylamino)thieno[3,2-c]pyridine-2-carbonitrile;
N¹-(3-chlorophenyl)-N⁶-(4-methoxybenzyl)-2,3-dihydro-1H-indene-1,6-diamine;
N¹-(3-chlorophenyl)-N⁶-methyl-N⁶-(4-(trifluoromethyl)benzyl)-2,3-dihydro-1H-indene-1,6-diamine;
N⁶-benzyl-N¹-(3-chlorophenyl)-N⁶-methyl-2,3-dihydro-1H-indene-1,6-diamine;
N-(3-bromophenyl)quinolin-4-amine;
6-chloro-N-(3-chlorophenyl)-2,3-dihydro-1H-inden-1-amine;
N-(3-chlorophenyl)-6-methyl-2,3-dihydro-1H-inden-1-amine;
3-(3-chlorophenylamino)-2,3-dihydro-1H-indene-5-carbonitrile;
N¹-(3-chlorophenyl)-N⁶-(2-ethoxyethyl)-2,3-dihydro-1H-indene-1,6-diamine;
N-(3-chlorophenyl)-6-methoxy-2,3-dihydro-1H-inden-1-amine;
6-chloro-N-(3-chlorophenyl)quinazolin-4-amine;
N-(3-chlorophenyl)phthalazin-1-amine;
N-(3-chlorophenyl)-4-methylnaphthalen-1-amine;
N-(3-chlorophenyl)-6-methoxy-2,3-dihydro-1H-inden-1-amine;
2-bromo-N-(3-chlorophenyl)thieno[3,2-c]pyridin-4-amine;
N-(3-fluorophenyl)-2,3-dihydro-1H-inden-1-amine;
(2R)-1-(3-chlorophenylamino)-2,3-dihydro-1H-inden-2-ol;
N¹-(3-chlorophenyl)-N⁶-methyl-N⁶-((R)-1-phenylethyl)-2,3-dihydro-1H-indene-1,6-diamine;
N-(3-chlorophenyl)-2,3-dihydro-1H-inden-4-amine;
N-(1-(3-chlorophenylamino)isoquinolin-3-yl)acetamide;
N-(3-chlorophenyl)-5,6,7,8-tetrahydronaphthalen-1-amine;
2-chloro-N-(3-chlorophenyl)thieno[3,2-c]pyridin-4-amine;
N-(3-chlorophenyl)-2-methylfuro[3,2-c]pyridin-4-amine;
N-(3-chlorophenyl)-6-fluoro-2,3-dihydro-1H-inden-1-amine;
6-(benzyl(methyl)amino)-2,3-dihydro-1H-inden-1-one;
N⁶-benzyl-N¹-(3-chlorophenyl)-N⁶-ethyl-2,3-dihydro-1H-indene-1,6-diamine;
3-amino-N-(3-chlorophenyl)-2-naphthamide;
3-chloro-N-(4-fluoro-1H-indazol-3-yl)benzamide;
N-(2,3-dihydro-1H-inden-4-yl)-7H-pyrrolo[2,3-d]pyrimidin-4-amine;
N-(3-chlorophenyl)-N-(6-(trifluoromethyl)quinolin-4-yl)acetamide;
4-(3-chlorophenylamino)-6-(trifluoromethyl)quinoline 1-oxide;
N¹-(3-chlorophenyl)-N⁶-(2-fluoro-4-methoxybenzyl)-N⁶-methyl-2,3-dihydro-1H-indene-1,6-diamine;
N¹-(3-chlorophenyl)-N⁶-(3-fluoro-4-methoxybenzyl)-N⁶-methyl-2,3-dihydro-1H-indene-1,6-diamine;
7-chloro-N-(3-chlorophenyl)-1H-indazol-3-amine;
5-chloro-N-(3-chlorophenyl)-1H-indazol-3-amine;
N-(3-chlorophenyl)-5,7-difluoro-1H-indazol-3-amine;
5-fluoro-N-(3-fluorophenyl)-1H-indazol-3-amine;
N-(3-fluorophenyl)-1H-indazol-3-amine;
5-fluoro-N-phenyl-1H-indazol-3-amine;
3-(3-chlorophenylamino)benzofuran-2-carboxamide;
5-chloro-N-(3-chlorophenyl)-1-methyl-1H-indazol-3-amine;
5-chloro-N-(3-fluorophenyl)-1-methyl-1H-indazol-3-amine;
N-(3-chlorophenyl)benzo[d]isoxazol-3-amine;
N-(3-chlorophenyl)-1H-indazol-3-amine;
N-(3-chlorophenyl)-1-methyl-1H-pyrazolo[3,4-b]pyridin-3-amine;
N-phenyl-1H-indazol-3-amine;
N,1-diphenyl-1H-indazol-3-amine;
N-(3-chlorophenyl)-5-fluoro-1H-indazol-3-amine;
N-(3-chlorophenyl)-5-methoxy-1-methyl-1H-indazol-3-amine;
N-(3-fluorophenyl)-5-methoxy-1-methyl-1H-indazol-3-amine;
4-chloro-N-(5-methoxy-1H-indazol-3-yl)benzamide;
N-(3-chlorophenyl)-6-phenylquinazolin-4-amine;
N-(3-chlorophenyl)-6-(4-fluorophenyl)quinazolin-4-amine;
N-(3-chlorophenyl)-6-(2,3-difluorophenyl)quinazolin-4-amine;
methyl 3-(4-(4-(3-chlorophenylamino)quinazolin-6-yl)phenyl)propanoate;
N-(3-chlorophenyl)-6-(thiophen-3-yl)quinazolin-4-amine;
N-(3-chlorophenyl)-8-methyl-9H-purin-6-amine;
6-(6-bromopyridin-3-yl)-N-(3-chlorophenyl)quinazolin-4-amine;
4-(4-(3-chlorophenylamino)quinazolin-6-yl)benzoic acid;
(3-chlorophenyl)(5-methoxy-1H-indazol-3-yl)methanone;
4-chloro-N-(5-methoxy-1H-indazol-3-yl)benzenesulfonamide;
N-(3-chlorophenyl)-6-(6-chloropyridin-3-yl)quinazolin-4-amine;
N-(3-chlorophenyl)-6-(3-fluorobiphenyl-4-yl)quinazolin-4-amine;
tert-butyl 4-(4-(3-chlorophenylamino)quinazolin-6-yl)phenylcarbamate;
N-(3-chlorophenyl)-6-ethylquinazolin-4-amine;
N-(3-chlorophenyl)-6-(4-chlorophenyl)quinazolin-4-amine;
6-(benzo[d][1,3]dioxol-5-yl)-N-(3-chlorophenyl)quinazolin-4-amine;
N-(3-chlorophenyl)-6-(2-fluoro-3-methoxyphenyl)quinazolin-4-amine;
N-(3-chlorophenyl)-6-(4-(morpholinosulfonyl)phenyl)quinazolin-4-amine;
1-(4-(4-(3-chlorophenylamino)quinazolin-6-yl)benzoyl)piperidin-4-one;
4-(4-(3-chlorophenylamino)quinazolin-6-yl)phenol;
N-(3-chlorophenyl)-6-(3-fluorophenyl)quinazolin-4-amine;
6-bromo-N-(3-chlorophenyl)quinazolin-4-amine;
N-(3-chlorophenyl)-6-(2-fluorophenyl)quinazolin-4-amine;
N-(3-chlorophenyl)-6-(thiophen-2-yl)quinazolin-4-amine;
N-(3-chlorophenyl)-6-(3,5-dimethylisoxazol-4-yl)quinazolin-4-amine;
N-(3-chlorophenyl)-6-cyclopropylquinazolin-4-amine;
N-(3-chlorophenyl)-1H-pyrazolo[3,4-d]pyrimidin-4-amine;
N-(3-chlorophenyl)-6-(5-methoxypyridin-3-yl)quinazolin-4-amine;
4-(4-(3-chlorophenylamino)quinazolin-6-yl)benzonitrile;
N-(3-chlorophenyl)-3-methyl-1H-pyrazolo[3,4-d]pyrimidin-4-amine;
N²,N⁴-bis(3-chlorophenyl)pyrido[2,3-d]pyrimidine-2,4-diamine;
N,1-bis(4-methoxybenzyl)-5-nitro-1H-indazol-3-amine;
N-(5-methoxy-1H-indazol-3-yl)thiophene-2-sulfonamide;
N-(3-chlorophenyl)-6-(6-fluoropyridin-3-yl)quinazolin-4-amine;
N-(3-chlorophenyl)-6-methylthieno[2,3-d]pyrimidin-4-amine;
N-(3-chlorophenyl)-6-(5-methylthiophen-2-yl)quinazolin-4-amine;
N-(3-chlorophenyl)-6-methylquinazolin-4-amine;
6-(5-bromothiophen-2-yl)-N-(3-chlorophenyl)quinazolin-4-amine;
N-(3-chlorophenyl)-7-methylthieno[3,2-d]pyrimidin-4-amine;
N-(3-chlorophenyl)-6-cyclohexenylquinazolin-4-amine;
N-(3-chlorophenyl)-6-(1H-pyrrol-2-yl)quinazolin-4-amine;
N-(3-chlorophenyl)-1H-pyrrolo[3,2-c]pyridin-4-amine;
N-(3-chlorophenyl)-5-nitro-1H-indazol-3-amine;
2-fluoro-N-(5-methoxy-1H-indazol-3-yl)benzenesulfonamide;
N-(3-chlorophenyl)quinolin-4-amine;
8-chloro-N-(3-chlorophenyl)quinolin-4-amine;
N-(3-chlorophenyl)-6-fluoroquinolin-4-amine;
N-(5-chloro-2-fluorophenyl)-7H-pyrrolo[2,3-d]pyrimidin-4-amine;
N-(3-chlorophenyl)-6-isopropylthieno[2,3-d]pyrimidin-4-amine;
N-(3-chlorophenyl)-2,3-dihydro-1H-inden-1-amine;
N-(3-chlorophenyl)-2-ethylfuro[3,2-c]pyridin-4-amine;
6-bromo-N-(3-chlorophenyl)-2,3-dihydro-1H-inden-1-amine;
N-(3-chlorophenyl)-6-morpholino-2,3-dihydro-1H-inden-1-amine;
N¹-(3-chlorophenyl)-N⁶-(2-(dimethylamino)ethyl)-2,3-dihydro-1H-indene-1,6-diamine;
N-(3-chlorophenyl)-2-methylquinolin-4-amine;
N-(3-chlorophenyl)-4-fluoronaphthalen-1-amine;
3-chloro-N-(3-chlorophenyl)isoquinolin-1-amine;
N-(3-chlorophenyl)-6-(trifluoromethyl)quinolin-4-amine;
N¹-(3-chlorophenyl)-N⁶-(4-methoxybenzyl)-N⁶-methyl-2,3-dihydro-1H-indene-1,6-diamine;
N-(3-chlorophenyl)-5-methoxy-1-(4-methoxybenzyl)-1H-indazol-3-amine;
N-(3-chlorophenyl)-5-methoxy-1-(4-methoxybenzyl)-1H-indazol-3-amine;
6-bromo-N-(3-chlorophenyl)-2,3-dihydro-1H-inden-1-amine;
N¹-(3-chlorophenyl)-N⁶-methyl-N⁶-phenethyl-2,3-dihydro-1H-indene-1,6-diamine;
N¹-(3-chlorophenyl)-N⁶-(4-ethylbenzyl)-N⁶-methyl-2,3-dihydro-1H-indene-1,6-diamine;
N⁶-benzyl-N¹-(3-chlorophenyl)-N⁶-methyl-2,3-dihydro-1H-indene-1,6-diamine;
N-(3-chlorophenyl)-1-methyl-1H-indole-3-carboxamide;
5-(difluoromethyl)-3-(naphthalen-2-yl)-1H-pyrazole;
N-(3-chlorophenyl)-2,3-dihydro-1H-inden-1-amine;
N-(3-chlorophenyl)-6,8-difluoroquinolin-4-amine;
N-(3-fluorophenyl)-7H-pyrrolo[2,3-d]pyrimidin-4-amine;
N-(3-chlorophenyl)-5H-pyrrolo[3,2-d]pyrimidin-4-amine;
N¹-(3-chlorophenyl)-N⁶-(4-(dimethylamino)benzyl)-N⁶-methyl-2,3-dihydro-1H-indene-1,6-diamine;
N⁶-(4-chlorobenzyl)-N¹-(3-chlorophenyl)-N⁶-methyl-2,3-dihydro-1H-indene-1,6-diamine;
N⁶-butyl-N¹-(3-chlorophenyl)-N⁶-methyl-2,3-dihydro-1H-indene-1,6-diamine;
N¹-(3-chloro-phenyl)-N⁶-methyl-N⁶-(1-methyl-piperidin-4-yl)-indan-1,6-diamine;
N³-(3-chloro-phenyl)-N⁵-indan-1-yl-1-methyl-1H-indazole-3,5-diamine;
(3-chloro-phenyl)-[5-(3-methoxy-benzyloxy)-1-methyl-1H-indazol-3-yl]-amine;
(3-chloro-phenyl)-(6-pyrazol-1-yl-indan-1-yl)-amine;
N³-(3-chloro-phenyl)-N⁵-isobutyl-1,N⁵-dimethyl-1H-indazole-3,5-diamine;
N³-(3-chloro-phenyl)-N⁵-(2-methoxy-ethyl)-1,N⁵-dimethyl-1H-indazole-3,5-diamine;
(R) —N¹-(3-chloro-phenyl)-N⁶-(4-methoxy-benzyl)-N⁶-methyl-indan-1,6-diamine;
(3,5-difluoro-phenyl)-[5-methoxy-1-(4-methoxy-benzyl)-1H-indazol-3-yl]-amine;
(3,5-dichloro-phenyl)-[5-methoxy-1-(4-methoxy-benzyl)-1H-indazol-3-yl]-amine;
(3,5-difluoro-phenyl)-(5-methoxy-1H-indazol-3-yl)-amine;
(3,5-dichloro-phenyl)-(5-methoxy-1H-indazol-3-yl)-amine;
N³-(3-chloro-phenyl)-N⁵-(2-methoxy-ethyl)-1-methyl-1H-indazole-3,5-diamine;
N¹-(3-chloro-phenyl)-N⁶-methyl-N⁶-pyridin-3-ylmethyl-indan-1,6-diamine;
N¹-(3-chloro-phenyl)-N⁶-methyl-N⁶-pyridin-4-ylmethyl-indan-1,6-diamine;
(3-chloro-phenyl)-(5-fluoro-1-methyl-1H-indazol-3-yl)-amine;
(3-chloro-phenyl)-(1-methyl-5-propoxy-1H-indazol-3-yl)-amine;
N¹-(3-chloro-phenyl)-N⁶-methyl-N⁶-thiophen-2-ylmethyl-indan-1,6-diamine;
(3-chloro-phenyl)-(1-methyl-1H-indazol-3-yl)-amine;
4-(3-chloro-phenylamino)-chromen-2-one;
1-[3-(3-chloro-phenylamino)-1-methyl-1H-indazol-5-yl]-ethanone;
N³-(3-chloro-phenyl)-N⁵-(2-methoxy-ethyl)-N⁵-methyl-1H-indazole-3,5-diamine;
N³-(3-chloro-phenyl)-N⁵-ethyl-1,N⁵-dimethyl-1H-indazole-3,5-diamine;
(3-Fluoro-phenyl)-(5-methoxy-1H-indazol-3-yl)-amine;
(2-chloro-pyridin-4-yl)-(5-methoxy-1H-indazol-3-yl)-amine;
(5-chloro-pyridin-3-yl)-(5-methoxy-1H-indazol-3-yl)-amine;
N¹-(3-chloro-phenyl)-N⁶-methyl-N⁶-thiophen-3-ylmethyl-indan-1,6-diamine;
1-[3-(3-chloro-phenylamino)-1-methyl-1H-indazol-5-yl]-ethanol;
(3-chloro-phenyl)-(1-methyl-5-methylsulfanyl-1H-indazol-3-yl)-amine;
2-[3-(3-chloro-phenylamino)-5-methoxy-indazol-1-yl]-acetamide;
(3-chloro-phenyl)-(5-methoxymethyl-1-methyl-1H-indazol-3-yl)-amine;
(3-chloro-phenyl)-(1-methyl-5-pyrrol-1-yl-1H-indazol-3-yl)-amine;
(3-chloro-phenyl)-(1-methyl-5-pyrazol-1-yl-1H-indazol-3-yl)-amine;
3-(3-chloro-phenylamino)-1-methyl-1H-indazole-5-carboxylic acid amide;
N³-(3-chloro-phenyl)-1,N⁵-dimethyl-1H-indazole-3,5-diamine;
{[3-(3-chloro-phenylamino)-indan-5-yl]-methyl-amino}-acetic acid ethyl ester;
1-[3-(3-chloro-phenylamino)-5-methoxy-indol-1-yl]-ethanone;
(3-chloro-phenyl)-(5-pyrazol-1-yl-1H-indazol-3-yl)-amine;
(2-chloro-pyrimidin-4-yl)-[5-methoxy-1-(4-methoxy-benzyl)-1H-indazol-3-yl]-amine;
(3-chloro-phenyl)-(5-methoxy-1H-indol-3-yl)-amine;
(5-chloro-benzo[d]isoxazol-3-yl)-(3-chloro-phenyl)-amine;
[3-(3-chloro-phenylamino)-1H-indazol-5-yl]-methanol;
(3-chloro-phenyl)-(1H-pyrazolo[4,3-b]pyridin-3-yl)-amine;
3-(3-chloro-phenylamino)-1H-indazole-5-carboxylic acid amide;
(3-chloro-phenyl)-[1-methyl-5-(4-methyl-pyrazol-1-yl)-1H-indazol-3-yl]-amine;
3-(3-chloro-phenylamino)-1H-indazole-5-carboxylic acid methyl ester;
indole-1-carboxylic acid (3-chloro-phenyl)-amide;
(3-bromo-phenyl)-(5-chloro-1-methyl-1H-indazol-3-yl)-amine;
(5-chloro-1-methyl-1H-indazol-3-yl)-(3-iodo-phenyl)-amine;
(3-chloro-phenyl)-(1H-pyrazolo[3,4-b]pyridin-3-yl)-amine;
3-(3-chloro-phenylamino)-1H-indazole-5-carbonitrile;
[5-(4-bromo-pyrazol-1-yl)-1-methyl-1H-indazol-3-yl]-(3-chloro-phenyl)-amine;
(7-chloro-4-fluoro-1H-indazol-3-yl)-(3-chloro-phenyl)-amine;
(5-chloro-1-methyl-1H-pyrazolo[3,4-b]pyridin-3-yl)-(3-chloro-phenyl)-amine;
(5-chloro-4-fluoro-1H-indazol-3-yl)-(3-chloro-phenyl)-amine;
(3-chloro-phenyl)-(5-methoxy-benzo[d]isoxazol-3-yl)-amine;
(3-chloro-phenyl)-(1H-pyrazolo[3,4-c]pyridin-3-yl)-amine;
[5-chloro-1-(4-methoxy-benzyl)-1H-pyrazolo[3,4-b]pyridin-3-yl]-(3-chloro-phenyl)-amine;
(3-chloro-phenyl)-(6-iodo-quinazolin-4-yl)-amine;
(3-chloro-phenyl)-(5-trifluoromethoxy-1H-indazol-3-yl)-amine;
(3-chloro-phenyl)-(5-chloro-1H-pyrazolo[3,4-b]pyridin-3-yl)-amine;
(3-chloro-phenyl)-(5-nitro-benzo[d]isothiazol-3-yl)-amine;
(3-chloro-phenyl)-(2-thiophen-2-yl-oxazolo[5,4-d]pyrimidin-7-yl)-amine;
(5-chloro-1-methyl-1H-thieno[2,3-c]pyrazol-3-yl)-(3-chloro-phenyl)-amine;
(3-chloro-phenyl)-[5-methoxy-1-(2-pyrrolidin-1-yl-ethyl)-1H-indazol-3-yl]-amine;
(3-chloro-phenyl)-(5-methoxy-1-methoxymethyl-1H-indazol-3-yl)-amine;
(3-chloro-phenyl)-[5-methoxy-1-(2-morpholin-4-yl-ethyl)-1H-indazol-3-yl]-amine;
(3-chloro-phenyl)-(6,8-diiodo-quinazolin-4-yl)-amine;
(3-chloro-phenyl)-(6-fluoro-chroman-4-yl)-amine;
1-[3-(3-chloro-phenylamino)-5-methoxy-indazol-1-yl]-2-morpholin-4-yl-ethanone;
3-[3-(3-chloro-phenylamino)-indazol-1-yl]-N,N-dimethyl-benzamide;
(3-chloro-phenyl)-(5-methoxy-1-pyridin-4-ylmethyl-1H-indazol-3-yl)-amine;
4-chloro-2-(7H-pyrrolo[2,3-d]pyrimidin-4-ylamino)-phenol;
(3-chloro-phenyl)-chroman-4-yl-amine;
(5-chloro-2-methoxy-phenyl)-(7H-pyrrolo[2,3-d]pyrimidin-4-yl)-amine;
(6-chloro-pyridin-2-yl)-(7H-pyrrolo[2,3-d]pyrimidin-4-yl)-amine;
(3-chloro-2-fluoro-phenyl)-(7H-pyrrolo[2,3-d]pyrimidin-4-yl)-amine;
[2-(2-bromo-phenyl)-ethyl]-(7H-pyrrolo[2,3-d]pyrimidin-4-yl)-amine;
[2-(4-bromo-phenyl)-ethyl]-(7H-pyrrolo[2,3-d]pyrimidin-4-yl)-amine;
(3-chloro-phenyl)-(5-methoxy-1-pyridin-3-ylmethyl-1H-indazol-3-yl)-amine;
(3-chloro-phenyl)-(5-methoxy-1-pyridin-2-ylmethyl-1H-indazol-3-yl)-amine;
(1-Benzyl-5-methoxy-1H-indazol-3-yl)-(3-chloro-phenyl)-amine;
(3-chloro-5-fluoro-phenyl)-(7H-pyrrolo[2,3-d]pyrimidin-4-yl)-amine;
(3-chloro-phenyl)-quinolin-5-yl-amine;
(3-chloro-phenyl)-(5-methoxy-1-thiazol-4-ylmethyl-1H-indazol-3-yl)-amine;
(3-chloro-phenyl)-(5-methoxy-1-pyridin-3-yl-1H-indazol-3-yl)-amine;
(3-chloro-4-fluoro-phenyl)-(7H-pyrrolo[2,3-d]pyrimidin-4-yl)-amine;
N⁴-(3-chloro-phenyl)-7H-pyrrolo[2,3-d]pyrimidine-2,4-diamine;
2-[3-(3-chloro-phenylamino)-5-methoxy-indazol-1-yl]-N-methyl-acetamide;
(5-bromo-1H-pyrazolo[3,4-b]pyridin-3-yl)-(3-chloro-phenyl)-amine;
(3-chloro-phenyl)-[1-(1-isobutyl-1H-pyrazol-4-yl)-5-methoxy-1H-indazol-3-yl]-amine;
(3-chloro-phenyl)-[5-methoxy-1-(tetrahydro-pyran-2-ylmethyl)-1H-indazol-3-yl]-amine;
(3-chloro-phenyl)-[5-methoxy-1-(2-pyrrol-1-yl-ethyl)-1H-indazol-3-yl]-amine;
(7H-pyrrolo[2,3-d]pyrimidin-4-ylamino)-acetic acid ethyl ester;
(3-chloro-phenyl)-[5-methoxy-1-(1-phenyl-ethyl)-1H-indazol-3-yl]-amine;
3-[3-(3-chloro-phenylamino)-5-methoxy-indazol-1-yl]-N,N-dimethyl-benzamide;
(3-chloro-phenyl)-[5-methoxy-1-(tetrahydro-pyran-4-ylmethyl)-1H-indazol-3-yl]-amine;
(3-chloro-phenyl)-[5-methoxy-1-(tetrahydro-furan-2-ylmethyl)-1H-indazol-3-yl]-amine;
(3-chloro-phenyl)-[5-methoxy-1-(5-methyl-isoxazol-3-ylmethyl)-1H-indazol-3-yl]-amine;
2-[3-(3-chloro-phenylamino)-5-methoxy-indazol-1-yl]-1-phenyl-ethanone;
2-[3-(3-chloro-phenylamino)-5-methoxy-indazol-1-yl]-1-(2-methoxy-phenyl)-ethanone;
2-[3-(3-chloro-phenylamino)-5-methoxy-indazol-1-yl]-1-(3-methoxy-phenyl)-ethanone;
2-[3-(3-chloro-phenylamino)-5-methoxy-indazol-1-yl]-1-phenyl-ethanol;
3-[3-(3-chloro-phenylamino)-5-methoxy-indazol-1-yl]-benzonitrile;
(3-chloro-phenyl)-[1-(2-diethylamino-ethyl)-5-methoxy-1H-indazol-3-yl]-amine;
(3-chloro-phenyl)-[5-methoxy-1-(2-methyl-thiazol-4-ylmethyl)-1H-indazol-3-yl]-amine;
2-{2-[3-(3-chloro-phenylamino)-5-methoxy-indazol-1-yl]-ethyl}-isoindole-1,3-dione;
1-{3-[3-(3-chloro-phenylamino)-5-methoxy-indazol-1-yl]-phenyl}-ethanone;
[5-bromo-1-(4-methoxy-benzyl)-1H-pyrazolo[3,4-b]pyridin-3-yl]-(3-chloro-phenyl)-amine;
1-{3-[3-(3-chloro-phenylamino)-5-methoxy-indazol-1-yl]-phenyl}-ethanol;
(1-Allyl-5-methoxy-1H-indazol-3-yl)-(3-chloro-phenyl)-amine;
(3-chloro-phenyl)-[1-(2-fluoro-benzyl)-5-methoxy-1H-indazol-3-yl]-amine;
(3-chloro-phenyl)-[1-(3-fluoro-benzyl)-5-methoxy-1H-indazol-3-yl]-amine;
(3-chloro-phenyl)-[1-(4-fluoro-benzyl)-5-methoxy-1H-indazol-3-yl]-amine;
2-[3-(3-chloro-phenylamino)-5-methoxy-indazol-1-yl]-1-phenyl-propan-1-one;
3-[3-(3-chloro-phenylamino)-5-methoxy-indazol-1-yl]-benzoic acid methyl ester;
(3-chloro-phenyl)-[1-(3,5-difluoro-benzyl)-5-methoxy-1H-indazol-3-yl]-amine;
(3-chloro-phenyl)-[5-methoxy-1-(2-trifluoromethoxy-benzyl)-1H-indazol-3-yl]-amine;
3-[3-(3-chloro-phenylamino)-5-methoxy-indazol-1-yl]-benzoic acid;
(1-sec-butyl-5-methoxy-1H-indazol-3-yl)-(3-chloro-phenyl)-amine;
(3-chloro-phenyl)-{1-[2-(4-fluoro-phenoxy)-ethyl]-5-methoxy-1H-indazol-3-yl}-amine;
(3-chloro-phenyl)-[1-(3-methanesulfonyl-phenyl)-5-methoxy-1H-indazol-3-yl]-amine;
3-[3-(3-chloro-phenylamino)-5-methoxy-indazol-1-yl]-benzaldehyde;
(3-chloro-phenyl)-{5-methoxy-1-[3-(pyrrolidine-1-sulfonyl)-phenyl]-1H-indazol-3-yl}-amine;
(3-chloro-phenyl)-{1-[2-(1H-indol-3-yl)-ethyl]-5-methoxy-1H-indazol-3-yl}-amine;
(1-benzo[1,2,5]thiadiazol-4-ylmethyl-5-methoxy-1H-indazol-3-yl)-(3-chloro-phenyl)-amine;
3-[3-(3-chloro-phenylamino)-5-methoxy-indazol-1-yl]-2,2-dimethyl-propan-1-ol;
(1-benzenesulfonyl-5-methoxy-1H-indazol-3-yl)-(3-chloro-4-fluoro-phenyl)-amine;
(3-chloro-4-fluoro-phenyl)-(5-methoxy-1H-indazol-3-yl)-amine;
3-[3-(3-chloro-phenylamino)-5-methoxy-indazol-1-yl]-N-methyl-benzamide;
(3-chloro-phenyl)-{5-methoxy-1-[3-(tetrahydro-pyran-2-yloxy)-propyl]-1H-indazol-3-yl}-amine;
3-[3-(3-chloro-phenylamino)-5-methoxy-indazol-1-yl]-N-isopropyl-benzamide;
3-[3-(3-chloro-phenylamino)-5-methoxy-indazol-1-yl]-propane-1,2-diol;
3-[3-(3-chloro-phenylamino)-5-methoxy-indazol-1-yl]-propan-1-ol;
(3-chloro-phenyl)-[5-methoxy-1-(2-phenyl-thiazol-4-ylmethyl)-1H-indazol-3-yl]-amine;
(3-chloro-phenyl)-[5-methoxy-1-(2-thiophen-2-yl-thiazol-4-ylmethyl)-1H-indazol-3-yl]-amine;
(3-chloro-4-fluoro-phenyl)-(6,8-difluoro-quinolin-4-yl)-amine;
(3-chloro-4-fluoro-phenyl)-(6-methyl-thieno[2,3-d]pyrimidin-4-yl)-amine;
(3-chloro-4-fluoro-phenyl)-isoquinolin-1-yl-amine;
(3-chloro-4-fluoro-phenyl)-(6-trifluoromethyl-quinolin-4-yl)-amine;
(3-chloro-4-fluoro-phenyl)-(6-fluoro-quinolin-4-yl)-amine;
(3-chloro-phenyl)-{1-[3-(isopropylamino-methyl)-phenyl]-5-methoxy-1H-indazol-3-yl}-amine;
(3-chloro-phenyl)-{5-methoxy-1-[(S)-1-(tetrahydro-pyran-2-yl)methyl]-1H-indazol-3-yl}-amine;
(3-chloro-phenyl)-{5-methoxy-1-[(R)-1-(tetrahydro-pyran-2-yl)methyl]-1H-indazol-3-yl}-amine;
3-[3-(3-chloro-phenylamino)-5-methoxy-indazol-1-yl]-N-(2,4-dimethoxy-benzyl)-benzamide;
3-[3-(3-chloro-phenylamino)-5-methoxy-indazol-1-yl]-N,N-dimethyl-benzenesulfonamide;
(3-chloro-phenyl)-(2-chloro-quinazolin-4-yl)-amine;
(3-chloro-phenyl)-[1-(2-[1,3]dioxolan-2-yl-ethyl)-5-methoxy-1H-indazol-3-yl]-amine;
4-(3-chloro-phenylamino)-naphthalene-1-carbonitrile;
N⁴-(3-chloro-phenyl)-N²-pentyl-quinazoline-2,4-diamine;
(3-chloro-phenyl)-(2-morpholin-4-yl-quinazolin-4-yl)-amine;
4-(3-chloro-phenylamino)-quinazoline-2-carboxylic acid ethyl ester; N²-benzyl-N⁴-(3-chloro-phenyl)-quinazoline-2,4-diamine;
N⁴-(3-chloro-phenyl)-N²-(2-methoxy-ethyl)-N²-methyl-quinazoline-2,4-diamine;
4-[3-(3-chloro-phenylamino)-5-methoxy-indazol-1-yl]-butan-2-one;
(3-chloro-2-fluoro-phenyl)-(6-trifluoromethyl-quinolin-4-yl)-amine;
(3-chloro-5-fluoro-phenyl)-(6-trifluoromethyl-quinolin-4-yl)-amine;
(5-chloro-2-fluoro-phenyl)-(6-trifluoromethyl-quinolin-4-yl)-amine;
(3-chloro-phenyl)-[5-methoxy-1-(2-methoxy-ethyl)-1H-indazol-3-yl]-amine;
2-[3-(3-chloro-phenylamino)-5-methoxy-indazol-1-yl]-N,N-dimethyl-acetamide;
3-[3-(3-chloro-phenylamino)-5-methoxy-indazol-1-yl]-butan-2-one;
2-[3-(3-chloro-phenylamino)-5-methoxy-indazol-1-yl]-propionitrile;
(3-chloro-phenyl)-[1-(3-ethanesulfonyl-phenyl)-5-methoxy-1H-indazol-3-yl]-amine;
(3-chloro-2-fluoro-phenyl)-isoquinolin-1-yl-amine;
(3-chloro-5-fluoro-phenyl)-isoquinolin-1-yl-amine;
[1-(2-amino-ethyl)-5-methoxy-1H-indazol-3-yl]-(3-chloro-phenyl)-amine;
5-methoxy-1-(4-methoxy-benzyl)-1H-pyrazolo[3,4-b]pyridin-3-ylamine;
4-[3-(3-chloro-phenylamino)-5-methoxy-indazol-1-yl]-butan-2-ol;
3-[3-(3-chloro-phenylamino)-5-methoxy-indazol-1-yl]-butan-2-ol;
(5-chloro-2-fluoro-phenyl)-isoquinolin-1-yl-amine;
N⁴-(3-chloro-phenyl)-N²-phenethyl-quinazoline-2,4-diamine;
N²-benzyl-N⁴-(3-chloro-phenyl)-N-methyl-quinazoline-2,4-diamine;
N⁴-(3-chloro-phenyl)-N²-(2-methoxy-ethyl)-quinazoline-2,4-diamine;
4-(3-chloro-phenylamino)-quinazoline-2-carboxylic acid;
(3-chloro-phenyl)-[1-(4-methanesulfonyl-phenyl)-5-methoxy-1H-indazol-3-yl]-amine;
3-[3-(3-chloro-phenylamino)-5-methoxy-indazol-1-yl]-benzenesulfonamide;
(3-chloro-phenyl)-(5-methoxy-1H-pyrazolo[3,4-b]pyridin-3-yl)-amine;
N⁴-(3-chloro-phenyl)-N²-phenyl-quinazoline-2,4-diamine; and
2-pyrazin-2-yl-thiazole-4-carboxylic acid (2-pyridin-2-yl-ethyl)-amide.

### 6.23. In Vitro Liver Microsomal Assay

This assay was modified from that described in Mark, G. et al. Journal of Pharmacology and Experimental Therapeutics 287:157-166 (1998), and employed commercially available rat liver microsomes to catalyze the conversion of ¹⁴C-radiolabeled dihomo-gamma-linolenic acid (DGLA) to radiolabeled arachidonic acid (AA). DGLA and AA are separated using argentation-TLC and are quantitated using a PhosphorImager.

The reaction was performed in a 200 μl volume and in a 96-well format with the following order of addition:

1) Forty μl of a ten-fold diluted (2 mg/ml in 100 mM sodium phosphate pH 7.4 with 250 mM sucrose, and 250 mM KCl) rat liver microsomes (Xenotech, Lenexa, Kans., USA).
2) Ten μl of a suitable concentration of inhibitory compound to be tested or DMSO as a control. Final DMSO concentration in 200 μl reaction was 0.25%.
3) One hundred fifty μl of buffer/substrate (100 mM sodium phosphate pH 7.4 with 250 mM sucrose, and 250 mM KCl, 1.3 mM ATP, 1.5 mM reduced glutathione, 0.06 mM reduced coenzyme A, 0.33 mM nicotinamide, 1 mM NADH, 5.4 mM MgCl₂, and 0.5 μl of radiolabeled DGLA (American Radiolabeled Chemicals Inc., Saint Louis, Mo., specific activity 55 mCi/mmol, concentration 0.1 mCi/ml) solution to initiate reaction. Final DGLA concentration was approximately 5 μM (0.05 μCi per reaction).

The reaction was allowed to proceed for 1 hour with gentle shaking at 37° C., after which the reaction was terminated by the addition of 200 μl of 2.5 N KOH (in 4 methanol: 1H₂O). This saponification step was continued for 2 hours at 65° C.

The reaction was then re-acidified by the addition of 280 μl of concentrated formic acid and 600 μl of hexane was added followed by thorough mixing. The deep-well plate was then centrifuged at 1000× g for 2 minutes allowing excellent separation of the aqueous (lower) and hexane (upper) layers. 300 μl of the hexane layer was removed and spotted on a Whatman K5 150 A silica gel TLC plate (20×20 cm, 250 μm thick, 19 channels) pre-coated by immersion in a 10% silver nitrate solution for 10-20 seconds and allowed to dry in air. TLC plates were stored in the dark and were activated at 110° C. for one hour just prior to use.

Argentation-TLC was performed using a solvent system containing chloroform:methanol:acetic acid:water (90:8:1:0.8). Chromatography continued for approximately 1 hour. Deasaturase activity was determined directly from the TLC plates by autoradiography using a PhosphorImager (Molecular Dynamics).

### 6.24. Cell-Based Assay

HepG2 cells were grown in Minimum Essential Media with GlutaMAX, with Earle's salts (Gibco 41090-036) plus 10% FBS, 1% GPS, 1% non Essential AA, 1% Sodium Pyruvate. The day before the assay, the media was changed to serum free media, and incubated overnight at 37° C. Cells were harvested and resuspended in serum free media containing 0.2% fatty acid free BSA.

One hundred eighty μl cells were transferred into each well (500,000 total cells) in a 96 deep well plate, then 10 μl compound were added at a suitable concentration. The cells were allowed to pre-incubate at 37° C. with the compound for 30 minutes. Ten μl dihomo-gamma-linolenic acid (DGLA, American Radiolabeled Chemicals Inc., Saint Louis, Mo., specific activity 55 mCi/mmol, concentration 0.1 mCi/ml) mix in 0.2% fatty acid free BSA and SF Media were added to cells. The final concentration of ¹⁴C DGLA was approximately 3 μM (0.025 μCi per well). The reaction was then incubated for 2 hours at 37° C. with gentle shaking, after which the cells were centrifuged at 1000× g for 10 minutes.

The media was removed and replaced with SF media+FAFBSA (200 μl). Two hundred μl 2.5 N KOH in 80% Methanol was added and saponified at 65° C. for 1 hour. Then, 280 μl formic acid and 600 μl hexane were added sequentially and mixed thoroughly. The resulting mixture was centrifuged at 1000× g for 2 minutes to separate aqueous and hexane layers. Two hundred μl of hexane layers was spotted on Whatman K5 150 A silica gel TLC plate (20×20 cm, 250 μm thick, 19 channels) pre-coated by immersion in a 10% silver nitrate solution for 10-20 seconds and activated at 110° C. for one hour. Argentation-TLC was performed as described above.

### 6.25. In Vivo Assay

This assay was modified from that described in Mark, G. et al. Journal of Pharmacology and Experimental Therapeutics 287:157-166 (1998). In it, ¹⁴C-DGLA was evaporated to dryness and resuspended into 18.2 mM Na₂CO. C57 wild-type mice were administered with compounds or vehicle at an appropriate time before DGLA were injection i.p. with 10 mCi DGLA per mouse. Two hours after DGLA injection, the mice were sacrificed, and their livers were quickly removed and frozen on dry ice. Half of each liver sample (˜0.5 g) was added into 8 ml of chloroform:methanol:water (1:2:0.3) and homogenized using Polytron for 30 seconds at room temperature. The homogenates were centrifuged for 10 minutes at 2500 rpm, and the supernatant was removed and placed in new tube. To the residue tissue, 4.6 ml of chloroform:methanol:water (1:2:0.8) were added and vortexed vigorously. The sample was centrifuged for 10 minutes at 2500 rpm, and the supernatant was removed and pooled with the first supernatant.

The pooled supernatant was diluted with 3.6 ml chloroform and then 3.6 ml water following by gentle mixing. The chloroform and methanol/water phase were separated by centrifugation at 2500 rpm for 10 minutes.

Three ml of chloroform layer were withdrawn, and one ml of 2.5N KOH was added. The resulting mixture was vortexed vigorously, and the sample saponified at 65° C. for 1.5 hours. After saponification, two ml of formic acid was added, followed by two ml of water and three ml of hexane. The mixture was vortexed vigorously, and then centrifuged for then minutes at 2500 rpm. The hexane layer was removed for TLC analysis as described above for the In Vitro Liver Microsomal Assay. Δ5-desaturase substrate (DGLA and derivative) and product (arachidonic acid and derivative) were quantitated by phosphoimaging.

### 6.26. Melanin-Concentrating Hormone Receptor Assay

Certain compounds were tested for their ability to inhibit human melanin-concentrating hormone (MCH₁) receptor by Cerep, Inc. (Redmond, Wash.), using an assay (Cerep SOP No. 1A164) adapted from Mac Donald, D., et al., Mol. Pharmacol., 58:217-225 (2000).

In the assay, the affinity of compounds for the agonist site of the human MCH₁ receptor in transfected CHO cells is determined by radioligand binding. Cell membrane homogenates (5 μg protein) are incubated for 60 min at 22° C. with 0.1 nM [¹²⁵I][Phe¹³,Tyr¹⁹]-MCH in the absence or presence of the test compound in a buffer containing 25 mM Hepes/Tris (pH 7.4), 5 mM MgCl₂, 1 mM CaCl₂ and 0.5% BSA. Nonspecific binding is determined in the presence of 0.1 μM MCH.

Following incubation, the samples are filtered rapidly under vacuum through glass fiber filters (GF/B, Packard) and rinsed several times with an ice-cold buffer containing 25 mM Hepes/Tris (pH 7.4), 500 mM NaCl, 5 mM MgCl₂, 1 mM CaCl₂ and 0.1% BSA using a 96-sample cell harvester (Unifilter, Packard). The filters are dried then counted for radioactivity in a scintillation counter (Topcount, Packard) using a scintillation cocktail (Microscint 0, Packard).

The results are expressed as a percent inhibition of the control radioligand specific binding. The standard reference compound is MCH, which is tested in each experiment at several concentrations to obtain a competition curve from which its IC₅₀ is calculated.

### 6.27. Calculating IC₅₀ Values

The IC₅₀ of a compound with regard to a given target is determined by fitting the relevant data, using the Levenburg Marquardt algorithm, to the equation:

y=A+((B−A)/(1+((C/x)̂D)))

wherein A is the minimum y value; B is the maximum y value; C is the IC₅₀; and D is the slope. The calculation of the IC₅₀ is performed using XLFit4 software (ID Business Solutions Inc., Bridgewater, N.J. 08807) for Microsoft Excel (the above equation is model 205 of that software).

6.28. Pharmacological Effects

Diet-induced obesity (DIO) mice were used to determine the effect of a potent Δ5-desaturase inhibitor. The mice were C57 wild type mice, weaned on a 45 percent fat diet, and maintained on that high fat diet for 30 weeks, after which their body composition was measured by qualitative magnetic resonance (QMR) (Bruker Minispec QMR Analyzer).

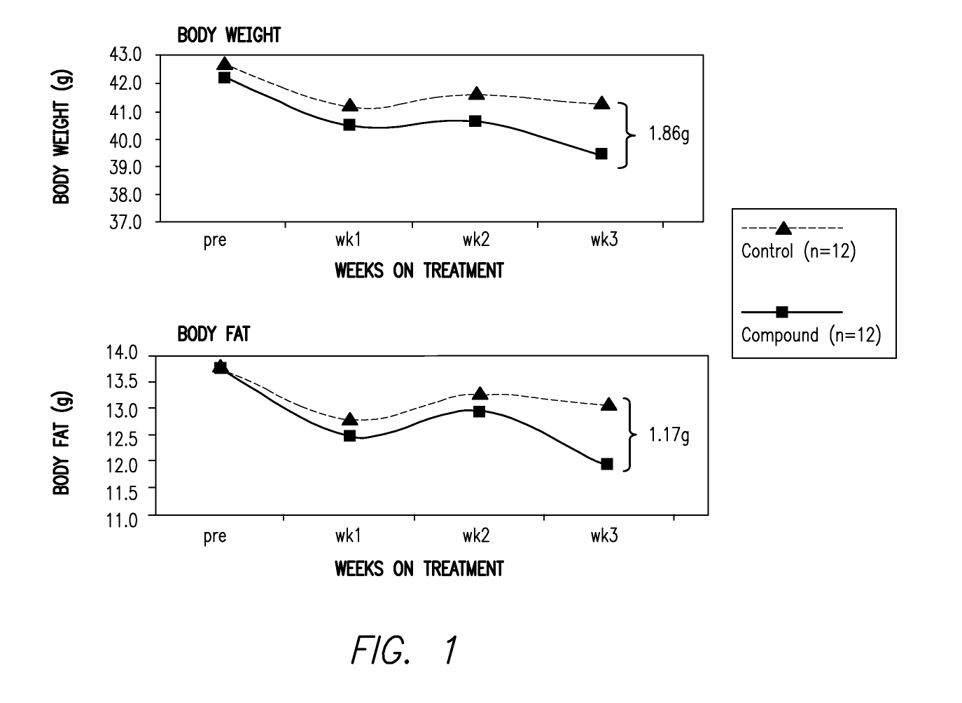

The Δ5-desaturase inhibitor was administered daily to twelve DIO mice for three weeks. The dose was administered orally: 300 mg/kg in the animals' feed, and an additional 100 mg/kg by oral gavage at 12:30 pm. Twelve control DIO mice were given a placebo gavage at the same time. The weight and percent body fat of the mice were determined by QMR. As shown in FIG. 1, administration of the compound decreased both the average weight and percent body fat of the mice. The food intake of dosed mice was the same as that of the control mice.

A separate six-week study was undertaken using the same Δ5-desaturase inhibitor administered to DIO mice under the same conditions as describe above. In this study, the mice lost both fat and muscle weight. Again, the food intake of dosed mice was the same as that of the control mice.

All cited publications, patents, and patent applications are herein incorporated by reference in their entireties.

1-119. (canceled)

120. A method of inhibiting Δ5-desaturase, which comprises contacting Δ5-desaturase with a compound of formula I: 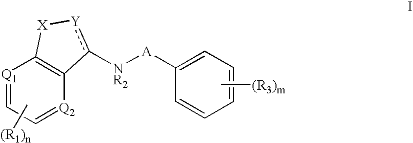
or a pharmaceutically acceptable salt or solvate thereof, wherein:
Q₁ is C or N;
Q₂ is C or N;
X is C(R₄R₅) or N(R₄);
Y is C(R₄), C(R₄R₅), N, or N(R₄);
A is nothing (i.e., NR₂ is directly attached to the optionally substituted phenyl moiety), CH₂, C(O), or SO₂;
each R₁ is independently OR_{1A}, N(R_{1A})₂, NC(O)R_{1A}, hydrogen, cyano, nitro, halo, or optionally substituted alkyl, aryl, alkylaryl, arylalkyl, heterocycle, alkylheterocycle or heterocyclealkyl;
each R_{1A} is independently hydrogen or optionally substituted alkyl, aryl, alkylaryl, arylalkyl, heterocycle, alkylheterocycle or heterocyclealkyl;
R₂ is hydrogen or optionally substituted alkyl;
each R₃ is independently OR_{3A}, N(R_{3A})₂, NC(O)R_{3A}, hydrogen, cyano, nitro, halo, or optionally substituted alkyl, aryl, alkylaryl, arylalkyl, heterocycle, alkylheterocycle or heterocyclealkyl;
each R_{3A} is independently hydrogen or optionally substituted alkyl, aryl, alkylaryl, arylalkyl, heterocycle, alkylheterocycle or heterocyclealkyl;
each R₄ is independently hydrogen or optionally substituted alkyl, aryl, alkylaryl, arylalkyl, heterocycle, alkylheterocycle or heterocyclealkyl;
each R₅ is independently hydrogen or optionally substituted alkyl, aryl, alkylaryl, arylalkyl, heterocycle, alkylheterocycle or heterocyclealkyl;
n is 1-4 if Q₁ and Q₂ are both C, 1-3 if one of Q₁ and Q₂ are N, or 1-2 if both Q₁ and Q₂ are N; and
m is 1-4.

121. (canceled)

122. (canceled)

123. A method of treating, preventing or managing a body composition disorder, which comprises administering to a patient in need thereof a therapeutically or prophylactically effective amount of a potent Δ5-desaturase inhibitor of formula I: 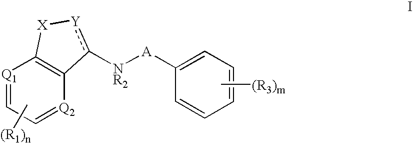
or a pharmaceutically acceptable salt or solvate thereof, wherein:
Q₁ is C or N;
Q₂ is C or N;
X is C(R₄R₅) or N(R₄);
Y is C(R₄), C(R₄R₅), N, or N(R₄);
A is nothing (i.e., NR₂ is directly attached to the optionally substituted phenyl moiety), CH₂, C(O), or SO₂;
each R₁ is independently OR_{1A}, N(R_{1A})₂, NC(O)R_{1A}, hydrogen, cyano, nitro, halo, or optionally substituted alkyl, aryl, alkylaryl, arylalkyl, heterocycle, alkylheterocycle or heterocyclealkyl;
each R_{1A} is independently hydrogen or optionally substituted alkyl, aryl, alkylaryl, arylalkyl, heterocycle, alkylheterocycle or heterocyclealkyl;
R₂ is hydrogen or optionally substituted alkyl;
each R₃ is independently OR_{3A}, N(R_{3A})₂, NC(O)R_{3A}, hydrogen, cyano, nitro, halo, or optionally substituted alkyl, aryl, alkylaryl, arylalkyl, heterocycle, alkylheterocycle or heterocyclealkyl;
each R_{3A} is independently hydrogen or optionally substituted alkyl, aryl, alkylaryl, arylalkyl, heterocycle, alkylheterocycle or heterocyclealkyl;
each R₄ is independently hydrogen or optionally substituted alkyl, aryl, alkylaryl, arylalkyl, heterocycle, alkylheterocycle or heterocyclealkyl;
each R₅ is independently hydrogen or optionally substituted alkyl, aryl, alkylaryl, arylalkyl, heterocycle, alkylheterocycle or heterocyclealkyl;
n is 1-4 if Q₁ and Q₂ are both C, 1-3 if one of Q₁ and Q₂ are N, or 1-2 if both Q₁ and Q₂ are N; and
m is 1-4.

124. (canceled)

125. (canceled)

126. The method of claim 123, wherein the metabolic or body composition disorder decreased glucose tolerance, diabetes (Type I or II), insulin resistance, non-insulin-dependent diabetes mellitus, obesity, or Syndrome X.

127. The method of claim 123, which does not substantially affect the appetite of the patient.

128. The method of claim 123, wherein the potent Δ5-desaturase inhibitor is of formula I(a):

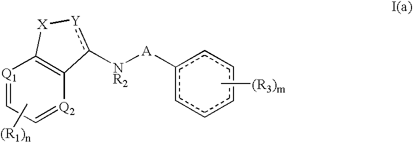

129. The method of claim 123, wherein the potent Δ5-desaturase inhibitor is of formula I(b):

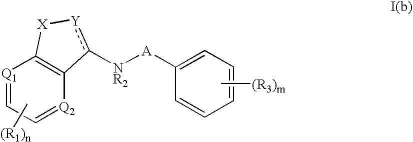

130. The method of claim 123, wherein the potent Δ5-desaturase inhibitor is of formula I(c):

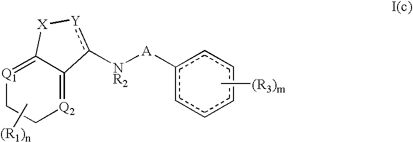

131. The method of claim 123, wherein the potent Δ5-desaturase inhibitor is of formula I(d):

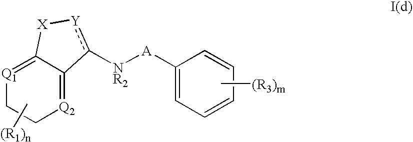

132. The method of claim 123, wherein the potent Δ5-desaturase inhibitor is of formula I(e):

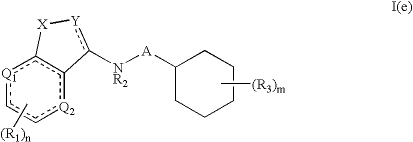

133. The method of claim 123, wherein both Q₁ and Q₂ are CR₁.

134. The method of claim 123, wherein X is N(R₄).

134. The method of claim 123, wherein Y is N or N(R₄).

135. The method of claim 123, wherein A is a bond.

136. The method of claim 123, wherein R₁ is not hydrogen.

137. The method of claim 123, wherein R₁ is halo or optionally substituted lower alkyl, aryl, or heteroaryl.

138. The method of claim 123, wherein R₁ is OR_{1A}.

139. The method of claim 138, wherein R_{1A} is lower alkyl.

140. The method of claim 123, wherein n is 1 or 2.

141. The method of claim 140, wherein n is 1.

142. The method of claim 123, wherein R₂ is hydrogen.

143. The method of claim 123, wherein R₂ is lower alkyl.

144. The method of claim 123, wherein R₃ is halo or optionally substituted lower alkyl, aryl, or heteroaryl.

145. The method of claim 144, wherein R₃ is optionally substituted lower alkyl.

146. The method of claim 144, wherein R₃ is halo.

147. The method of claim 123, wherein R₃ is OR_{3A}.

148. The method of claim 147, wherein R_{3A} is lower alkyl.
